(19) United States
(12) Patent Application Publication
(10) Pub. No.: US 2022/0054630 A1
(43) Pub. Date: Feb. 24, 2022

(54) MODIFIED HEPATITIS C VIRUS E2 GLYCOPROTEINS AND METHODS OF USE THEREOF

(71) Applicant: University of Maryland, College Park, College Park, MD (US)

(72) Inventors: Brian G. Pierce, College Park, MD (US); Thomas R. Fuerst, College Park, MD (US); Roy A. Mariuzza, College Park, MD (US); Steven K.H. Foung, College Park, MD (US); Zhen-Yong Keck, College Park, MD (US)

(21) Appl. No.: 17/405,884

(22) Filed: Aug. 18, 2021

Related U.S. Application Data
(60) Provisional application No. 63/067,135, filed on Aug. 18, 2020.

(51) Int. Cl. A61K 39/29; C07K 14/18; A61P 31/14

(52) U.S. Cl. CPC A61K 39/29; A61P 31/14; C07K 14/1833

(57) ABSTRACT

Disclosed are modified HCV E2 glycoproteins. Disclosed are modified HCV E2 glycoproteins comprising an antigenic domain D, wherein the modified HCV E2 glycoproteins comprise one or more amino acid alterations in the antigenic domain D, wherein at least one amino acid alteration is a proline substitution. In some aspects, the proline substitution occurs at position 445 based on the amino acid numbering of HCV strain H77. Disclosed are modified HCV E2 glycoproteins comprising an antigenic domain A, wherein the antigenic domain A comprises an N-glycan sequon substitution. In some aspects, the N-glycan sequon substitution results in an Asn-Xaa-Ser or Asn-Xaa-Thr substitution, wherein Xaa is any amino acid except proline. Also disclosed are methods of using the disclosed modified HCV E2 glycoproteins, such as methods of inducing an immune response in a subject, methods of treating a subject, and methods of increasing antigenicity of HCV E2 glycoprotein.

### CROSS-REFERENCE TO RELATED APPLICATION

This application claims the benefit of U.S. Provisional Patent Application No. 63/067,135 filed on Aug. 18, 2020, and hereby is incorporated by reference herein in its entirety.

### STATEMENT REGARDING FEDERALLY SPONSORED RESEARCH

This invention was made with government support under Grant Numbers R01 AI132213 and R21AI126582 awarded by the National Institute of Health. The government has certain rights in this invention.

### REFERENCE TO SEQUENCE LISTING

The Sequence Listing submitted Aug. 18, 2021 as a text file named “36429_0028U2_Sequence_Listing.txt,” created on Aug. 18, 2021, and having a size of 29,784 bytes is hereby incorporated by reference pursuant to 37 C.F.R. § 1.52(e)(5).

### BACKGROUND

Hepatitis C virus (HCV) infection is a major global disease burden, with 71 million individuals, or approximately 1% of the global population, chronically infected worldwide, and 1.75 million new infections per year. Chronic HCV infection can lead to cirrhosis and hepatocellular carcinoma, the leading cause of liver cancer, and in the United States HCV was found to surpass HIV and 59 other infectious conditions as a cause of death. While the development of direct-acting antivirals has improved treatment options considerably, several factors impede the effective use of antiviral treatment such as the high cost of antivirals, viral resistance, and occurrence of reinfections after treatment cessation, and lack of awareness of infection in many individuals since HCV infection is considered a silent epidemic.

Despite decades of research resulting in several HCV vaccine candidates tested in vivo and in clinical trials, no approved HCV vaccine is available. There are a number of barriers to the development of an effective HCV vaccine, including the high mutation rate of the virus which leads to viral quasi-species in individuals and permits active evasion of T cell and B cell responses. Escape from the antibody response by HCV includes mutations in the envelope glycoproteins, as observed in vivo in humanized mice, studies in chimpanzee models, and through analysis of viral isolates from human chronic infection. This was also clearly demonstrated during clinical trials of a monoclonal antibody, HCV1, which in spite of its targeting a conserved epitope on the viral envelope, failed to eliminate the virus, as viral variants with epitope mutations emerged under immune pressure and dominated the rebounding viral populations in all treated individuals.

There have been a number of successful structure-based vaccine designs for variable viruses such as influenza, HIV, and RSV where rationally designed immunogens optimize presentation of key conserved epitopes, mask sites using N-glycans, or stabilize conformations or assembly of the envelope glycoproteins. Recent studies have reported use of several of these strategies in the context of HCV glycoproteins, including removal or modification of N-glycans to improve epitope accessibility, removal of hypervariable regions, or presentation of key conserved epitopes on scaffolds. However, such studies have been relatively limited compared with other viruses, in terms of design strategies employed and number of designs tested, and immunogenicity, studies have not shown convincing improvement of glycoprotein designs over native glycoproteins in terms of neutralization potency or breadth, with the possible exception of an HVR-deleted high molecular weight form of the E2 glycoprotein that was tested in guinea pigs. Therefore, development of an effective, preventative vaccine for HCV is necessary to reduce the burden of infection and transmission, and for global elimination of HCV.

### BRIEF SUMMARY

Disclosed are modified HCV E2 glycoproteins.

Disclosed are modified HCV E2 glycoproteins comprising an antigenic domain D, wherein the modified HCV E2 glycoproteins comprise one or more amino acid alterations in the antigenic domain D, wherein at least one amino acid alteration is a proline substitution. In some aspects, the proline substitution occurs at position 445 based on the amino acid numbering of HCV strain H77.

Disclosed are modified HCV E2 glycoproteins comprising an antigenic domain A, wherein the antigenic domain A comprises an N-glycan sequon substitution. In some aspects, the N-glycan sequon substitution results in an Asn-Xaa-Ser or Asn-Xaa-Thr substitution, wherein Xaa is any amino acid except proline.

Disclosed are polynucleotides comprising a nucleic acid sequence capable of encoding one or more of the disclosed modified HCV glycoproteins.

Disclosed are vectors comprising any of the polynucleotides disclosed herein.

Disclosed are compositions comprising one or more of the modified HCV E2 glycoproteins described herein and a pharmaceutically acceptable carrier thereof.

Also disclosed are cells or cell lines comprising the compositions, vectors, polynucleotides or modified HCV E2 glycoproteins disclosed herein.

Disclosed are methods of increasing HCV E2 glycoprotein antigenicity in a subject in need thereof comprising administering a composition comprising one or more of the modified HCV E2 glycoproteins described herein, wherein the increase in HCV E2 glycoprotein antigenicity is an increase in antigenic domain D antigenicity.

Disclosed are method of decreasing HCV E2 glycoprotein antigenicity in a subject in need thereof comprising administering a composition comprising one or more of the modified HCV E2 glycoproteins described herein, wherein the decrease in HCV E2 glycoprotein antigenicity is a decrease in antigenic domain A antigenicity.

Disclosed are methods of inducing an immune response in a subject in need thereof comprising administering to the subject in need thereof a composition comprising one or more of the modified HCV E2 glycoproteins disclosed herein.

Also disclosed are methods of treating a subject having HCV or at risk of being infected with HCV comprising administering to the subject a composition comprising one or more of the modified HCV E2 glycoproteins disclosed herein.

Disclosed are methods of generating neutralizing antibodies (nAbs) to the antigenic domain D of HCV in a subject in need thereof comprising administering to the subject in need thereof a composition comprising one or more of the modified HCV E2 glycoproteins described herein.

Also disclosed are methods for immunizing a subject in need thereof comprising administering to the subject in need thereof a composition comprising one or more of the modified HCV E2 glycoproteins disclosed herein.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive example aspects of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A and 1B show structure-based design of E2 to stabilize and mask epitopes. FIG. 1A shows the design of the E2 front layer. (top) Antigenic domain B-D supersite (also referred to as “antigenic region 3”) is indicated with a circle on the E2 core X-ray structure (Kong L, et al., 2013. Science 342:1090-4) with modeled N-glycans shown as gray sticks. Ramachandran plot analysis for proline-like backbone conformation (middle) and structural modeling of proline substitution structural and energetic effects (bottom) were performed using RosettaDesign and the HC84.26.5D-AS434 epitope complex structure (Keck Z Y, et al., Hepatology 64:1922-1933). HC84.26.5D HMAb is shown in surface representation, epitope is shown in cartoon representation with selected mutant residue (H445P) shown as sticks and labeled. (FIG. 1B) Design of the E2 back layer. (top) Antigenic domain A (circled) was targeted for design and is shown on the E2 core structure (middle) Computational N-glycan scanning of antigenic domain A residues was performed to identify substitutions to mask its surface with designed N×S and N×T sequons. (bottom) Modeling of sequon mutants was performed in Rosetta (Kortemme T, et al., Sci STKE 2004:pl2), followed by modeling of N-glycan structures in the Glyprot Server (Bohne-Lang A, and von der Lieth C W. 2005, Nucleic acids research 33:W214-9). Modeled N-glycan design at Y632 (Y632N-G634S) is circled and shown in stick representation, in the context of the E2 core structure.

FIG. 2 shows antigenic characterization of E2 designs using ELISA. Designs were cloned and expressed in the context of E1E2 as previously described (Pierce B G, et al., 2016. Proc Natl Acad Sci USA 113:E6946-E6954) and tested for binding to a panel of HMAbs that target E2 antigenic domain A (CBH-4G, CBH-4B), B (HC-1), C (CBH-7), D (HC84.28, HC84.24, HC84.26), and E (HC33.1, HC33.4), at concentrations of 1 μg/ml, and 5 μg/ml. Binding was tested to wild-type H77C E1E2, and compared with designs ΔHVR1 (E2 residues 384-407 deleted), ΔHVR1₄₁₁ (E2 residues 384-410 deleted), H445P, F627NT (F627N-V629T), R630NT (R630N-Y632T), K628NS (K628N-R630S), and Y632NS (Y632N-G634S). Asterisks denote designs that were tested in the context of ΔHVR1₄₁₁ (E2 residues 384-410 deleted) rather than full length E1E2.

FIGS. 3A and 3B show antigenic characterization of sE2 designs H445P and Y632NS using biolayer interferometry (BLI). (FIG. 3A) Measured binding of broadly neutralizing monoclonal antibody HC84.26.WH.5DL to E2 design H445P compared to wild-type soluble E2 (sE2). (FIG. 3B) Measured binding of non-neutralizing monoclonal antibody CBH-4G to E2 design Y632NS (Y632N-G634S) compared to wild-type soluble E2 (sE2). Steady-state binding curve fits are shown, which were used to determine binding dissociation constants (K_{d}) values.

FIGS. 4A and 4B show immunized serum recognition of E2 and two E2 epitopes. FIG. 4A shows immunized sera were tested using ELISA for binding to soluble H77C E2 (sE2) and linear epitopes from antigenic domain E (AS412, aa 410-425) and antigenic domain D (AS434, aa 434-446). Serum binding was tested at successive three-fold dilutions starting at 1:60, and values are reported as endpoint titers. FIG. 4B shows binding of peptides to control monoclonal antibodies HC33.1 (Keck Z, et al., J Virol 87:37-51), AP33 (Owsianka A, et al., J Virol 79:11095-104), and HC84.26.WH.5DL (Ringe R P, et al., 2017, J Virol 91).

FIG. 5 shows serum binding competition with monoclonal antibodies. Serum inhibition of binding by biotinylated monoclonal antibodies at a concentration of 1 μg/ml was tested at the serum dilutions shown, using ELISA. The monoclonal antibodies tested for serum competition target E2 antigenic domains A (CBH-4G), B (HC-1), and D (HC84.26).

FIGS. 6A-6D show immunized serum binding to recombinant E1E2 and HCV pseudoparticles (HCVpp). Immunized sera were tested for binding to (FIG. 6A) H77C E1E2, and HCVpp representing (FIG. 6B) H77C, (FIG. 6C) UKNP1.18.1, and (FIG. 6D) J6 isolates using ELISA. Serum binding was tested at three-fold dilutions starting at 1:100, and values are reported as endpoint titers. Murine sera with binding levels lower than the endpoint OD value at the minimum dilution (1:100) have titers shown as 50. P-values between group endpoint titer values were calculated using Kruskal-Wallis analysis of variance with Dunn's multiple comparison test, and significant p-values between sE2 control and sE2 design groups are shown (*: p≤0.05; ****: p≤0.0001).

FIG. 7 shows a comparison of concentrated HCV pseudoparticle (HCVpp) binding of immunized mouse sera from sE2 wild-type and H445P immunization. A preparation enriched in H77C HCVpps was tested for binding to pooled murine sera from sE2 wild-type and H445P groups using ELISA, for sera from Day 42 and Day 56. Best-fit curves are shown and were used to calculate EC50 values.

FIG. 8 shows binding of concentrated HCV pseudoparticles (HCVpps), pseudotyped with H77C E1E2, to monoclonal antibodies. Binding measurements were performed using ELISA with antibodies targeting E2 (HCV1, HC84.26.WH.5DL, AR3A), E1E2 (AR4A, AR5A) and a negative control antibody (CA45).

FIG. 9 shows serum neutralization of homologous (H77C) and heterologous HCVpp. Immunized murine serum neutralization was tested using HCV pseudoparticles (HCVpps) representing H77C as well as six heterologous isolates. Neutralization for four HCVpp representing isolates with resistant phenotypes are shown on the right, as indicated. Neutralization titers are represented as serum dilution levels required to reach 50% virus neutralization (ID50), calculated by curve fitting in Graphpad Prism software. Serum dilutions were performed as two-fold dilutions starting at 1:64, and minimum dilution levels (corresponding to 1:64) are indicated as dotted lines for reference. Murine sera with low (calculated ID50<10) or incalculable ID50 values due to low or background levels of neutralization (observed only for some mice for J6 HCVpp neutralization) have ID50 shown as 10. P-values between group ID50 values were calculated using Kruskal-Wallis analysis of variance with Dunn's multiple comparison test, and significant p-values between sE2 control and sE2 design groups are shown (*: p≤0.05; **: p≤0.01).

FIGS. 10A, 10B, and 10C show an analysis of correlations in immunogenicity and antigenicity measurements. (FIG. 10A) Pairs of datasets of serum HCVpp neutralization (IC50) and antigen binding (endpoint titer) measurements were tested for Pearson correlations on an individual mouse level (42 points per dataset), and top correlations between datasets are shown. Pearson correlations were calculated using log-transformed ID50 and endpoint titer values. (FIG. 10B) UKNP2.4.1 versus UKNP1.18.1 serum neutralization (ID50), with best-fit line in red, and calculated correlation (r) and p-value (p) shown. (FIG. 10C) Correlations between antigen binding (K_{d}) and immunogenicity measurements for corresponding antigen group (group geometric mean ID50 or endpoint titer) were calculated, and most significant correlations are shown. Pearson correlations were calculated using negated log-transformed K_{d} and log-transformed titer values. (D) UKNP1.18.1 group serum neutralization (ID50) versus HC84.26.WH.5DL HMAb affinity (K_{d}), with best-fit line in red, and calculated correlation (r) and p-value (p) shown. The log-scale x-axis for HC84.26.WH.5DL K_{d} is shown with reversed scale, in accordance with the polarity of the calculated correlation. For (A) and (C), correlation p-values are shown above each bar (*: p≤0.05; **: p≤0.01, ***: p≤0.001, ****: p≤0.0001).

FIG. 11 is a table showing backbone structure and proline mutant analysis of antigenic domain D residues.

FIG. 12 is a table showing calculated surface accessibility of E2 residues in antigenic domain A.

FIG. 13 is a table showing antigenic and biophysical characterization of E2 designs.

FIG. 14 is a table showing percentage of occupancy for engineered N-glycan at position 632, determined by mass spectrometry.

FIG. 15 is a table showing a panel of viral isolates used in neutralization assays.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of aspects of the present invention which will be limited only by the appended claims.

It must be noted that as used herein and in the appended claims, the singular forms “a”, “an”, and “the” include plural reference unless the context clearly dictates otherwise. Thus, for example, reference to “a glycoprotein” includes a plurality of such glycoproteins, reference to “the glycoprotein” is a reference to one or more glycoproteins and equivalents thereof known to those skilled in the art, and so forth.

The term “hepatitis C virus” or “HCV”, as used herein, refers to any one of a number of different genotypes and isolates of hepatitis C virus. Thus, “HCV” encompasses any of a number of genotypes, subtypes, or quasispecies, of HCV, including, e.g., genotype 1, 2, 3, 4, 6, 7, 8, etc. and subtypes (e.g., 1a, 1b, 2a, 2b, 3a, 4a, 4c, etc.), and quasispecies. Representative HCV genotypes and isolates include: the “Chiron” isolate HCV-1, H77, J6, Con1, isolate 1, BK, EC1, EC10, HC-J2, HC-J5; HC-J6, HC-J7, HC-J8, HC-JT, HCT18, HCT27, HCV-476, HCV-KF, “Hunan”, “Japanese”, “Taiwan”, TH, type 1, type 1a, H77 type 1b, type 1c, type 1d, type 1e, type 1f, type 10, type 2, type 2a, type 2b, type 2c, type 2d, type 2f, type 3, type 3a, type 3b, type 3g, type 4, type 4a, type 4c, type 4d, type 4f, type 4h, type 4k, type 5, type 5a, type 6 and type 6a.

The HCV genome comprises a 5′-untranslated region that is followed by an open reading frame (ORF) that codes for about 3,010 amino acids. The ORF runs from nucleotide base pair 342 to 8,955 followed by another untranslated region at the 3′ end. The amino acids are subdivided into ten proteins in the order from 5′ to 3′ as follows: C; E1; E2; NS1; NS2; NS3; NS4 (a and b); and NS5 (a and b). These proteins are formed from the cleavage of the larger polyprotein by both host and viral proteases. The C, E1, and E2 proteins are structural and the NS1-NS5 proteins are nonstructural proteins. The C region codes for the core nucleocapsid protein. E1 and E2 are glycosylated envelope proteins that coat the virus. NS2 may be a zinc metalloproteinase. NS3 is a helicase. NS4a functions as a serine protease cofactor involved in cleavage between NS4b and NS5a. NS5a is a serine phosphoprotein whose function is unknown. The NS5b region has both RNA-dependent RNA polymerase and terminal transferase activity.

As used herein, the term “subject” or “patient” can be used interchangeably and refer to any organism to which a protein or composition of this invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates; and the like.

The term “percent (%) identity” can be used interchangeably herein with the term “percent (%) homology” and refers to the level of nucleic acid or amino acid sequence identity when aligned with a wild type sequence using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 80, 85, 90, 95, 98% or more sequence identity to a given sequence, e.g., the coding sequence for any one of the inventive proteins, as described herein. Exemplary computer programs which can be used to determine identity between two sequences include, but are not limited to, the suite of BLAST programs, e.g., BLASTN, BLASTX, and TBLASTX, BLASTP and TBLASTN, publicly available on the Internet. See also, Altschul, et al., 1990 and Altschul, et al., 1997. Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62matrix. (See, e.g., Altschul, S. F., et al., Nucleic Acids Res.25:3389-3402, 1997.) A preferred alignment of selected sequences in order to determine“% identity” between two or more sequences, is performed using for example, the CLUSTAL-W program in Mac Vector version 13.0.7, operated with default parameters, including an open gap penalty of 10.0, an extended gap penalty of 0.1, and a BLOSUM 30 similarity matrix.

Amino acid alterations such as substitutions, deletions, insertions or any combination thereof may be used to arrive at a final derivative, variant, or analog. Generally, these changes are done on a few nucleotides to minimize the alteration of the molecule. However, larger changes may be tolerated in certain circumstances.

Generally, the nucleotide identity between individual variant sequences can be at least 90%, 91%, 92%, 93%, 94%, 95%, 96%, 97%, 98%, 99%, or 100%. Thus, a “variant sequence” can be one with the specified identity to a parent or reference sequence (e.g. wild-type sequence) of the invention that comprises one or more amino acid alterations, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. In some aspects, a variant hepatitis C virus (HCV) E2 glycoprotein can be one or more of the modified hepatitis C virus (HCV) E2 glycoproteins disclosed herein. For example, a modified hepatitis C virus (HCV) E2 glycoprotein can be a sequence that contains 1, 2, or 3, 4 amino acid base changes as compared to the parent or reference sequence of the invention, and shares or improves biological function, specificity and/or activity of the parent sequence. Thus, a modified hepatitis C virus (HCV) E2 glycoprotein can be one with the specified identity to the parent sequence of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. The variant sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of a reference sequence (e.g. wild-type sequence or E2 protein sequence).

The terms “variant” and “mutant” or “modified” can be used interchangeably. As used herein, the term “variant” refers to a modified nucleic acid or protein which displays the same characteristics when compared to a reference nucleic acid or protein sequence. A modified hepatitis C virus (HCV) E2 glycoprotein can be at least 65, 70, 75, 80, 85, 90, 95, or 99 percent homologous to a reference sequence. In some aspects, a reference sequence can be a wild type hepatitis C virus (HCV) E2 glycoprotein nucleic acid sequence or a wild type hepatitis C virus (HCV) E2 glycoprotein protein sequence. Variants can also include nucleotide sequences that are substantially similar to sequences of E2 disclosed herein. A “variant” or “variant thereof” can mean a difference in some way from the reference sequence other than just a simple deletion of an N- and/or C-terminal amino acid residue or residues. Where the variant includes a substitution of an amino acid residue, the substitution can be considered conservative or non-conservative. Variants can include at least one substitution and/or at least one addition, there may also be at least one deletion. Variants can also include one or more non-naturally occurring residues.

As used herein an amino acid “substitution” refers to the replacement of one amino acid residue by a different amino acid residue. The substituted amino acid may be any of the 20 amino acids commonly found in human proteins, as well as atypical or non-naturally occurring amino acids. A substitution of an amino acid residue can be considered conservative or non-conservative. Conservative substitutions are those within the following groups: Ser, Thr, and Cys; Leu, ILe, and Val; Glu and Asp; Lys and Arg; Phe, Tyr, and Trp; and Gln, Asn, Glu, Asp, and His. In some aspects, the substitution can be a non-naturally occurring substitution. For example, the substitution may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH₂(CH₂)ₙCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

As used herein, the term “wild-type” refers to a gene or protein which has the characteristics of that gene or protein when isolated from a naturally-occurring source. For example, a wild type HCV E2 glycoprotein has the characteristics of the E2 glycoprotein from a naturally occurring HCV genotype such as H77.

By “treat” is meant to administer a protein, nucleic acid, or composition of the invention to a subject, such as a human or other mammal (for example, an animal model), that has an increased susceptibility for developing infection with HCV or that has an infection with HCV, in order to prevent or delay a worsening of the effects of the disease or condition, or to partially or fully reverse the effects of the disease or condition.

By “prevent” is meant to minimize the chance that a subject who has an increased susceptibility for developing an infection with HCV actually develops the infection or disease or otherwise develops a cause of symptom thereof.

As used herein, the terms “administering” and “administration” refer to any method of providing a disclosed peptide, composition, or a pharmaceutical preparation to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration for a disclosed composition or a disclosed protein so as to treat a subject or induce an immune response. In an aspect, the skilled person can also alter or modify an aspect of an administering step so as to improve efficacy of a disclosed protein, nucleic acid, composition, or a pharmaceutical preparation.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Modified Hepatitis C Virus (HCV) Glycoprotein

1. Hepatitis C Virus (HCV) E2 Glycoprotein

Disclosed are modified HCV E2 glycoproteins. In some aspects, modified HCV E2 glycoproteins are any HCV E2 glycoprotein having at least about 70, 75, 80, 85, 90, 95, or 99% identity, but not 100% identity, to a wild type HCV E2 glycoprotein from any of the known HCV genotypes and/or subtypes and comprising one or more amino acid alterations in the antigenic domain D. For example, disclosed are modified HCV E2 glycoproteins having at least about 70, 75, 80, 85, 90, 95, or 99% identity, but not 100% identity, to the H77 (Genbank AF009606) genotype of HCV and comprising one or more amino acid alterations in the antigenic domain D. Thus, disclosed are variants of HCV E2 glycoprotein.

In some instances, a modified HCV E2 glycoprotein can have at least about 70, 75, 80, 85, 90, 95, or 99% identity, but not 100% identity, to amino acid residues 384-746 of NCBI Accession No. NP_671491.1 (HCV strain H77). Amino acid residues 384-746 of NCBI Accession No. NP_671491.1 are ETHVTGGSAGRTTAGLVGLLTPGAKQNIQLINTNGSWHINSTALNCNESLNTGWLAG LFYQHKFNSSGCPERLASCRRLTDFAQGWGPISYANGSGLDERPYCWHYPPRPCGIV PAKSVCGPVYCFTPSPVVVGTTDRSGAPTYSWGANDTDVFVLNNTRPPLGNWFGCT WMNSTGFTKVCGAPPCVIGGVGNNTLLCPTDCFRKHPEATYSRCGSGPWITPRCMV DYPYRLWHYPCTINYTIFKVRMYVGGVEHRLEAACNWTRGERCDLEDRDRSELSPL LLSTTQWQVLPCSFTTLPALSTGLIHLHQNIVDVQYLYGVGSSIASWAIKWEYVVLLF LLLADARVCSCLWMMLLISQAEA (SEQ ID NO:1). SEQ ID NO:1 is the HCV E2 glycoprotein of the membrane bound form of the HCV E1E2 glycoprotein.

In some aspects, disclosed are modified HCV E2 glycoproteins comprising an amino acid sequence with at least about 70, 75, 80, 85, 90, 95, or 99% identity to SEQ ID NO: 1 and comprising one or more amino acid alterations in the antigenic domain D.

In some aspects, a modified HCV E2 glycoprotein is a full length HCV E2 glycoprotein. In some aspects, a modified HCV E2 glycoprotein is a full length HCV E2 glycoprotein and comprising one or more amino acid alterations in the antigenic domain D. In some aspects, a modified HCV E2 glycoprotein can have a length of from about 200 amino acids (aa) to about 250 aa, from about 250 aa to about 275 aa, from about 275 aa to about 300 aa, from about 300 aa to about 325 aa, from about 325 aa to about 350 aa, or from about 350 aa to about 365 aa. In some aspects, a modified HCV E2 glycoprotein can have a length of from about 200 amino acids (aa) to about 250 aa, from about 250 aa to about 275 aa, from about 275 aa to about 300 aa, from about 300 aa to about 325 aa, from about 325 aa to about 350 aa, or from about 350 aa to about 365 aa and comprising one or more amino acid alterations in the antigenic domain D.

Disclosed are modified HCV E2 glycoproteins comprising an antigenic domain D, wherein the modified HCV E2 glycoproteins comprise one or more amino acid alterations in the antigenic domain D. In some aspects, an amino acid alteration can be an amino acid substitution, deletion, or addition.

In an exemplary embodiment, carrier proteins represented by virus capsid proteins that have the capability to self-assemble into virus-like particles (VLPs) are utilized in combination with the disclosed modified HCV E2 glycoproteins or modified membrane bound HCV E1E2 glycoproteins. Examples of VLPs used as peptide carriers are hepatitis B virus surface antigen and core antigen, hepatitis E virus particles, polyoma virus, bovine papilloma virus, and the like.

In another embodiment, the disclosed modified HCV E2 glycoproteins or modified membrane bound HCV E1E2 glycoproteins are coupled to one of a number of carrier molecules, known to those of skill in the art. A carrier protein must be of sufficient size for the immune system of the subject to which it is administered to recognize its foreign nature and develop antibodies to it.

In some cases the carrier molecule is directly coupled to the disclosed modified HCV E2 glycoproteins or modified membrane bound HCV E1E2 glycoproteins. In other cases, there is a linker molecule inserted between the carrier molecule and the disclosed modified HCV E2 glycoproteins or modified membrane bound HCV E1E2 glycoproteins. For example, the coupling reaction may require a free sulfhydryl group on the peptide. In such cases, an N-terminal cysteine residue is added to the peptide when the peptide is synthesized. In an exemplary embodiment, traditional succinimide chemistry is used to link the peptide to a carrier protein. Methods for preparing such peptide:carrier protein conjugates are generally known to those of skill in the art and reagents for such methods are commercially available (e.g., from Sigma Chemical Co.). Generally about 5-30 peptide molecules are conjugated per molecule of carrier protein.

Any of the disclosed modified HCV E2 glycoproteins or modified membrane bound HCV E1E2 glycoproteins can be combined with other viral subunits to form an attenuated live virus or replication-defective virus. In some aspects, the disclosed modified HCV E2 glycoproteins or modified membrane bound HCV E1E2 glycoproteins can be combined with other elements to form nanoparticles carrying the disclosed modified HCV E2 glycoproteins or modified membrane bound HCV E1E2 glycoproteins.

In some aspects, the disclosed modified HCV E2 glycoproteins or modified membrane bound HCV E1E2 glycoproteins can be combined with one or more of the modifications to E2 described in U.S. Pat. No. 9,732,121, herein incorporated by reference in its entirety.

i. Proline Substitution

Disclosed herein are modified hepatitis C virus (HCV) E2 glycoproteins comprising an antigenic domain D, wherein the modified hepatitis C virus (HCV) E2 glycoprotein comprises one or more amino acid alterations in the antigenic domain D. In some aspects, an amino acid alteration is an amino acid substitution. Disclosed are modified HCV E2 glycoproteins comprising an antigenic domain D, wherein the modified HCV E2 glycoproteins comprise one or more amino acid alterations in the antigenic domain D, wherein at least one amino acid alteration is a proline substitution. In some aspects, the proline substitution stabilizes an antibody-bound conformation of the antigenic domain D.

As disclosed herein, the modified hepatitis C virus (HCV) E2 glycoproteins disclosed herein can be from any HCV strain or genotype, including HCV genotype H77. With regard to the position of a particular mutation and the numbering used herein, the numbering refers to the numbering based on the HCV genotype H77. While other HCV genotypes may vary in sequence from the HCV strain H77, the positions of the disclosed amino acid alterations can be identified in any non-H77 HCV genotypes (and therefore non-H77 HCV E2 and E1E2 sequences) using tools such as those found at https://hcv.lanl.gov/content/sequence/NEW ALIGN/align.html where a person of skill in the art, when provided with the information and guidance from the instant application can utilize the “H77 Coordinates”, as a means to identify and correlate the described positions (e.g. amino acid alterations) to specify the sites in non-H77 HCV sequences. For example, a person of skill in the art when provided with the information and guidance from the instant application can utilize the “H77 Coordinates”, to identify the positions corresponding to HCV genotype H77 positions 445, 632, and 634 in other HCV genotype amino acid sequences.

As provided herein, disclosed are modified HCV E2 glycoproteins comprising an antigenic domain D, wherein the modified HCV E2 glycoproteins comprise one or more amino acid alterations in the antigenic domain D, wherein at least one amino acid alteration is a proline substitution. In some aspects, the proline substitution occurs at position 445 based on the amino acid numbering of HCV strain H77. For example, a proline substitution at position 445 based on the amino acid numbering of HCV strain H77 is equivalent to a proline substitution at position 445 of strain JFH-1 (genotype 2a), which is an asparagine residue, or position 445 of strain S52 (genotype 3a), which is a histidine residue. However, in some aspects, position 445 based on the amino acid numbering of HCV strain H77 can be equivalent to a position different than 445 in a different strain or genotype. In some aspects, a proline substitution at position 445 based on the amino acid numbering of HCV strain H77 is equivalent to a proline substitution at position 62 of SEQ ID NO:1, which is the H77 E2 amino acid sequence. Position 445 is based on the full genomic polyprotein sequence of H77 whereas position 62 is based on just the HCV E2 glycoprotein amino acid sequence of H77 (SEQ ID NO. 1). In some aspects, the proline substitution is a substitution of histidine (at position 445 of H77 or at a position corresponding with position 445 of H77) with proline. In other words, in some aspects, the proline substitution corresponds to an H445P substitution in SEQ ID NO:1. In some aspects, the proline substitution is a substitution of asparagine, arginine, or tyrosine (at a position corresponding with position 445 of the HCV E2 glycoprotein amino acid sequence of H77) with proline. In some aspects, the proline substitution is a substitution of any amino acid (at a position corresponding with position 445 of H77) with proline.

In some aspects, the modified HCV E2 glycoprotein comprises SEQ ID NO:2. In some aspects, the modified HCV E2 glycoprotein consists of SEQ ID NO:2. SEQ ID NO:2 is the H77 E2 glycoprotein comprising a H445P substitution as shown below: ETHVTGGSAGRTTAGLVGLLTPGAKQNIQLINTNGSWHINSTALNCNESLNTGWLAG LFYQPKFNSSGCPERLASCRRLTDFAQGWGPISYANGSGLDERPYCWHYPPRPCGIV PAKSVCGPVYCFTPSPVVVGTTDRSGAPTYSWGANDTDVFVLNNTRPPLGNWFGCT WMNSTGFTKVCGAPPCVIGGVGNNTLLCPTDCFRKHPEATYSRCGSGPWITPRCMV DYPYRLWHYPCTINYTIFKVRMYVGGVEHRLEAACNWTRGERCDLEDRDRSELSPL LLSTTQWQVLPCSFTTLPALSTGLIHLHQNIVDVQYLYGVGSSIASWAIKWEYVVLLF LLLADARVCSCLWMMLLISQAEA (SEQ ID NO:2). A H445P substitution is shown in bold.

In some aspects, the modified HCV E2 glycoprotein comprises a sequence with 70, 75, 80, 85, 90, 95, or 99% identity to SEQ ID NO:2, wherein the sequence comprises a H445P substitution as compared to SEQ ID NO:2. In other words, the modified HCV E2 glycoprotein comprises a sequence with 70, 75, 80, 85, 90, 95, or 99% identity to SEQ ID NO:2, wherein the sequence comprises at least the H445P substitution. Thus, the 70, 75, 80, 85, 90, 95, or 99% identity can be based on an alteration somewhere other than position 445 of SEQ ID NO:2. In some aspects, modified hepatitis C virus (HCV) E2 glycoproteins are disclosed comprising at least a proline substitution at position 445 of E2 as compared to SEQ ID NO:1.

In some aspects, the antigenic domain D of the modified HCV E2 glycoprotein retains the ability to bind to an antibody specific to the antigenic domain D. For example, the H445P mutation present in SEQ ID NO:2 compared to SEQ ID NO:1 retains the ability of the modified HCV E2 glycoprotein to bind to an antibody specific to the antigenic domain D. In some aspects, the antibody specific to the antigenic domain D is HC84.1 or HC84.26. Therefore, in some aspects the antigenic domain D of a modified HCV E2 glycoprotein retains the ability to bind to HC84.1 or HC84.26.

In some aspects, the modified HCV E2 glycoproteins disclosed herein comprise an amino acid alteration in the antigenic D domain, wherein the amino acid alteration is a deletion of amino acids 384-407 as compared to SEQ ID NO:1. In some aspects, the modified HCV E2 glycoproteins disclosed herein comprise an amino acid alteration in the antigenic D domain, wherein the amino acid alteration is a deletion of amino acids 384-407 as compared to SEQ ID NO:1 and further comprise a proline substitution disclosed herein. For example, disclosed herein are modified hepatitis C virus (HCV) E2 glycoproteins comprising an antigenic domain D, wherein the modified hepatitis C virus (HCV) E2 glycoprotein comprises one or more amino acid alterations in the antigenic domain D, wherein the amino acid alteration in the antigenic D domain is a deletion of amino acids 384-407, wherein the modified hepatitis C virus (HCV) E2 glycoprotein further comprises a H445P substitution as compared to SEQ ID NO:1.

In some aspects, the modified HCV E2 glycoproteins disclosed herein are soluble. In some aspects, the soluble portion of the modified E2 glycoprotein of H77 is residues 384-661 of SEQ ID NO:1. Thus, in some aspects, the soluble portion of the modified E2 glycoprotein of H77, or secreted E2, is:

(SEQ ID NO: 3)
ETHVTGGSAGRTTAGLVGLLTPGAKQNIQLINTNGSWHINSTALNCN
ESLNTGWLAGLFYQHKFNSSGCPERLASCRRLTDFAQGWGPISYANG
SGLDERPYCWHYPPRPCGIVPAKSVCGPVYCFTPSPVVVGTTDRSGA
PTYSWGANDTDVFVLNNTRPPLGNWFGCTWMNSTGFTKVCGAPPCVI
GGVGNNTLLCPTDCFRKHPEATYSRCGSGPWITPRCMVDYPYRLWHY
PCTINYTIFKVRMYVGGVEHRLEAACNWTRGERCDLEDRDRSE.

In some aspects, the modified HCV E2 glycoprotein comprises the soluble portion of an HCV E2 glycoprotein comprising a proline substitution corresponding to position 445 of SEQ ID NO:1. In some aspects, the soluble portion of an HCV E2 glycoprotein, or secreted HCV E2 glycoprotein, is the secreted form of a HCV E2 glycoprotein SEQ ID NO:2. For example, the sequence of the soluble portion of the HCV E2 glycoprotein of H77, or secreted form of the HCV E2 glycoprotein of SEQ ID NO:2, can be ETHVTGGSAGRTTAGLVGLLTPGAKQNIQLINTNGSWHINSTALNCNESLNTGWLAG LFYQPKFNSSGCPERLASCRRLTDFAQGWGPISYANGSGLDERPYCWHYPPRPCGIV PAKSVCGPVYCFTPSPVVVGTTDRSGAPTYSWGANDTDVFVLNNTRPPLGNWFGCT WMNSTGFTKVCGAPPCVIGGVGNNTLLCPTDCFRKHPEATYSRCGSGPWITPRCMV DYPYRLWHYPCTINYTIFKVRMYVGGVEHRLEAACNWTRGERCDLEDRDRSE (SEQ ID NO:4). The H445P mutation is shown in bold. In some aspects, the modified HCV E2 glycoprotein comprises the amino acid sequence of SEQ ID NO:4. In some aspects, the modified HCV E2 glycoprotein consists of the amino acid sequence of SEQ ID NO:4.

ii. N-Glycan Sequon Substitution

N-glycosylation functions by modifying appropriate asparagine residues of proteins with oligosaccharide structures, thus influencing their properties and bioactivities. Disclosed are modified HCV E2 glycoproteins comprising an N-glycosylation in their antigenic domain A which blocks or decreases binding of antibodies to the antigenic domain A. In some aspects, the decrease in binding of antibodies to antigenic domain A of HCV E2 glycoprotein can result in an increased binding to antigenic domain D which can provide a neutralizing effect.

Disclosed are modified HCV E2 glycoproteins comprising an antigenic domain A, wherein the antigenic domain A comprises an N-glycan sequon substitution. An N-glycan sequon is a sequence of consecutive amino acids in a protein that can serve as the attachment site for an N-glycan. In some aspects, the N-glycan sequon substitution is in the antigenic domain A of SEQ ID NO:1. In some aspects, the N-glycan sequon substitution is in the antigenic domain A of an amino acid sequence with 70, 75, 80, 85, 90, 95 or 99% identity to SEQ ID NO:1.

In some aspects, the N-glycan sequon substitution results in an Asn-Xaa-Ser or Asn-Xaa-Thr substitution, wherein Xaa is any amino acid except proline.

In some aspects, the N-glycan sequon substitution corresponds to position 632-634 as compared to SEQ ID NO:1. For example, disclosed are N-glycan sequon substitutions at position 632 and 634, based on the amino acid numbering of H77, that result in an asparagine at position 632 and a serine or threonine at position 634. In some aspects, the N-glycan sequon substitution corresponds to position 630-632 as compared to SEQ ID NO:1. In some aspects, the N-glycan sequon substitution corresponds to position 628-630 as compared to SEQ ID NO:1. In some aspects, the N-glycan sequon substitution corresponds to position 627-629 as compared to SEQ ID NO:1.

In some aspects, the N-glycan sequon substitution is Y632N-G634S as compared to SEQ ID NO:1. For example, a modified HCV E2 glycoprotein comprising the N-glycan sequon substitution of Y632N-G634S compared to SEQ ID NO:1 comprises the sequence of ETHVTGGSAGRTTAGLVGLLTPGAKQNIQLINTNGSWHINSTALNCNESLNTGWLAG LFYQHKFNSSGCPERLASCRRLTDFAQGWGPISYANGSGLDERPYCWHYPPRPCGIV PAKSVCGPVYCFTPSPVVVGTTDRSGAPTYSWGANDTDVFVLNNTRPPLGNWFGCT WMNSTGFTKVCGAPPCVIGGVGNNTLLCPTDCFRKHPEATYSRCGSGPWITPRCMV DYPYRLWHYPCTINYTIFKVRMNVSGVEHRLEAACNWTRGERCDLEDRDRSELSPL LLSTTQWQVLPCSFTTLPALSTGLIHLHQNIVDVQYLYGVGSSIASWAIKWEYVVLLF LLLADARVCSCLWMMLLISQAEA (SEQ ID NO:5). A Y632N-G634S substitutions are shown in bold. IN some aspects, a modified HCV E2 glycoprotein comprising the N-glycan sequon substitution of Y632N-G634S compared to SEQ ID NO:1 consists of SEQ ID NO:5.

In some aspects, the modified HCV E2 glycoprotein is the soluble portion of an HCV E2 glycoprotein comprising an N-glycan substitution in antigenic domain A. In some aspects, the modified HCV E2 glycoprotein is the soluble portion of an HCV E2 glycoprotein comprising an N-glycan substitution in antigenic domain A corresponding to positions 632 and 634 of SEQ ID NO:1. In some aspects, the soluble portion of an HCV E2 glycoprotein, or secreted HCV E2 glycoprotein, is the secreted form of SEQ ID NO:5. For example, the sequence of the soluble portion of the HCV E2 glycoprotein of H77, or secreted form of SEQ ID NO:5, can be ETHVTGGSAGRTTAGLVGLLTPGAKQNIQLINTNGSWHINSTALNCNESLNTGWLAG LFYQHKFNSSGCPERLASCRRLTDFAQGWGPISYANGSGLDERPYCWHYPPRPCGIV PAKSVCGPVYCFTPSPVVVGTTDRSGAPTYSWGANDTDVFVLNNTRPPLGNWFGCT WMNSTGFTKVCGAPPCVIGGVGNNTLLCPTDCFRKHPEATYSRCGSGPWITPRCMV DYPYRLWHYPCTINYTIFKVRMNVSGVEHRLEAACNWTRGERCDLEDRDRSE (SEQ ID NO:9). A Y632N-G634S substitutions are shown in bold.

In some aspects, the N-glycan sequon substitution is R630N-Y632T as compared to SEQ ID NO:1. In some aspects, the N-glycan sequon substitution is K628N-R630S as compared to SEQ ID NO:1. In some aspects, the N-glycan sequon substitution is F627N-V629T as compared to SEQ ID NO:1.

In some aspects, the N-glycan sequon substitution is in the antigenic domain A of an amino acid sequence with 70, 75, 80, 85, 90, 95 or 99% identity to SEQ ID NO:5, wherein the antigenic domain A comprises the N-glycan sequon substitution of Y632N-G634S as compared to SEQ ID NO:1. In other words, the modified HCV E2 glycoprotein comprises a sequence with 70, 75, 80, 85, 90, 95, or 99% identity to SEQ ID NO:4, wherein the N-glycan sequon substitution in the antigenic domain A comprises the an N at position 632 and an S at position 634 wherein the numbers correspond to the numbering of H77. Thus, the reason for the less than 100% identity is due to an alteration in the sequence somewhere other than the Y632N-G634S mutations corresponding to positions 632 and 634 of SEQ ID NO:1.

In some aspects, the N-glycan sequon substitution is in the antigenic domain A of an amino acid sequence with 70, 75, 80, 85, 90, 95 or 99% identity to SEQ ID NO:5, wherein the antigenic domain A comprises the N-glycan sequon substitution of R630N-Y632T as compared to SEQ ID NO:1. In other words, the modified HCV E2 glycoprotein comprises a sequence with 70, 75, 80, 85, 90, 95, or 99% identity to SEQ ID NO:4, wherein the N-glycan sequon substitution in the antigenic domain A comprises the an N at position 630 and an T at position 632 wherein the numbers correspond to the numbering of H77.

In some aspects, the N-glycan sequon substitution is in the antigenic domain A of an amino acid sequence with 70, 75, 80, 85, 90, 95 or 99% identity to SEQ ID NO:5, wherein the antigenic domain A comprises the N-glycan sequon substitution of K628N-R630S as compared to SEQ ID NO:1. In other words, the modified HCV E2 glycoprotein comprises a sequence with 70, 75, 80, 85, 90, 95, or 99% identity to SEQ ID NO:4, wherein the N-glycan sequon substitution in the antigenic domain A comprises the an N at position 628 and a S at position 630 wherein the numbers correspond to the numbering of H77.

In some aspects, the N-glycan sequon substitution is in the antigenic domain A of an amino acid sequence with 70, 75, 80, 85, 90, 95 or 99% identity to SEQ ID NO:5, wherein the antigenic domain A comprises the N-glycan sequon substitution of F627N-V629T as compared to SEQ ID NO:1. In other words, the modified HCV E2 glycoprotein comprises a sequence with 70, 75, 80, 85, 90, 95, or 99% identity to SEQ ID NO:4, wherein the N-glycan sequon substitution in the antigenic domain A comprises the an N at position 627 and a T at position 629 wherein the numbers correspond to the numbering of H77.

In some aspects, the N-glycan sequon substitutions can be combined with any of the amino acid alterations in the antigenic D domain of E2 described herein. For example, in some aspects, disclosed are modified HCV E2 glycoproteins comprising a proline substitution at the amino acid corresponding to position 445 of SEQ ID NO:1 and an arginine substitution and serine or threonine substitution at the amino acids corresponding to positions 632 and 634, respectively, of SEQ ID NO:1.

2. Membrane Bound E1E2 Glycoproteins

The envelope of HCV contains two glycoproteins, E1 and E2, that are encoded as part of the HCV polyprotein expressed in infected liver cells. This polyprotein is processed in the endoplasmic reticulum (ER) by signal peptidases and cellular glycosylation machinery to produce the mature E1E2 complex. These glycoproteins are membrane-anchored via their C-terminal transmembrane domains (TMDs), resulting in a membrane bound E1 E2 (mbE1E2) complex.

Furthermore, E1E2 assembly has been proposed to form a trimer of heterodimers mediated by hydrophobic C-terminal transmembrane domains (TMDs) and interactions between E1 and E2 ectodomains. These glycoproteins are necessary for viral entry and infection, as E2 attaches to the CD81 and SR-B1 co-receptors as part of a multistep entry process on the surface of hepatocytes. Neutralizing antibody responses to HCV infection target epitopes in E1, E2, or the E1E2 heterodimer. HCV envelope proteins, E1 and E2, can thus form heterodimers on the viral surface and can be critical for HCV cell entry. Disclosed herein are membrane bound HCV E1E2 heterodimers (e.g. modified membrane bound HCV E1 E2 heterodimers) comprising an HCV E1 glycoprotein and a modified HCV E2 glycoprotein, wherein the modified HCV E2 glycoprotein comprises an antigenic domain D, wherein the modified HCV E2 glycoproteins comprise one or more amino acid alterations in the antigenic domain D.

In some aspects, E2 glycoprotein can be the membrane bound form of the modified HCV E2 glycoprotein as provided in SEQ ID NO:2. In some aspects, the modified HCV E2 glycoprotein present in the disclosed modified membrane bound E1E2 glycoproteins is a modified E2 glycoprotein comprising a proline substitution in position 445 compared to SEQ ID NO:1.

In some aspects, disclosed are modified membrane bound E1E2 glycoproteins comprising the HCV E2 glycoprotein of SEQ ID NO:2. In some aspects, the wild type H77 membrane bound HCV E1E2 glycoprotein comprises the sequence of: YQVRNSSGLYHVTNDCPNSSIVYEAADAILHTPGCVPCVREGNASRCWVAVTPTVA TRDGKLPTTQLRRHIDLLVGSATLCSALYVGDLCGSVFLVGQLFTFSPRRHWTTQDC NCSIYPGHITGHRMAWDMMMNWSPTAALVVAQLLRIPQAIMDMIAGAHWGVLAGI AYFSMVGNWAKVLVVLLLFAGVDAETHVTGGSAGRTTAGLVGLLTPGAKQNIQLIN TNGSWHINSTALNCNESLNTGWLAGLFYQHKFNSSGCPERLASCRRLTDFAQGWGPI SYANGSGLDERPYCWHYPPRPCGIVPAKSVCGPVYCFTPSPVVVGTTDRSGAPTYSW GANDTDVFVLNNTRPPLGNWFGCTWMNSTGFTKVCGAPPCVIGGVGNNTLLCPTDC FRKHPEATYSRCGSGPWITPRCMVDYPYRLWHYPCTINYTIFKVRMYVGGVEHRLE AACNWTRGERCDLEDRDRSE (SEQ ID NO:6). In some aspects, the modified membrane bound HCV E1E2 glycoprotein comprises an HCV E1 glycoprotein and a modified HCV E2 glycoprotein, wherein the modified HCV E2 glycoprotein comprises a proline substitution in the antigenic domain D, and wherein the modified membrane bound HCV E1E2 glycoprotein comprises the sequence of: YQVRNSSGLYHVTNDCPNSSIVYEAADAILHTPGCVPCVREGNASRCWVAVTPTVA TRDGKLPTTQLRRHIDLLVGSATLCSALYVGDLCGSVFLVGQLFTFSPRRHWTTQDC NCSIYPGHITGHRMAWDMMMNWSPTAALVVAQLLRIPQAIMDMIAGAHWGVLAGI AYFSMVGNWAKVLVVLLLFAGVDAETHVTGGSAGRTTAGLVGLLTPGAKQNIQLIN TNGSWHINSTALNCNESLNTGWLAGLFYQPKFNSSGCPERLASCRRLTDFAQGWGPI SYANGSGLDERPYCWHYPPRPCGIVPAKSVCGPVYCFTPSPVVVGTTDRSGAPTYSW GANDTDVFVLNNTRPPLGNWFGCTWMNSTGFTKVCGAPPCVIGGVGNNTLLCPTDC FRKHPEATYSRCGSGPWITPRCMVDYPYRLWHYPCTINYTIFKVRMYVGGVEHRLE AACNWTRGERCDLEDRDRSE (SEQ ID NO:7). A H445P mutation is shown in bold, wherein the H445P numbering is based on the residue positions of E2 in H77 (SEQ ID NO:1).

In some aspects, the modified membrane bound HCV E1E2 glycoprotein comprises an HCV E1 glycoprotein and a modified HCV E2 glycoprotein, wherein the modified membrane bound HCV E1E2 glycoprotein has 70, 75, 80, 85, 90, 95, or 99% identity to SEQ ID NO:7, and wherein modified membrane bound HCV E1E2 glycoprotein retains the proline at position 254 of SEQ ID NO:7.

In some aspects, the modified membrane bound HCV E1E2 glycoprotein comprises an HCV E1 glycoprotein and a modified HCV E2 glycoprotein, wherein the modified HCV E2 glycoprotein comprises an N-glycan sequon substitution, and wherein the modified membrane bound HCV E1E2 glycoprotein comprises the sequence of: YQVRNSSGLYHVTNDCPNSSIVYEAADAILHTPGCVPCVREGNASRCWVAVTPTVA TRDGKLPTTQLRRHIDLLVGSATLCSALYVGDLCGSVFLVGQLFTFSPRRHWTTQDC NCSIYPGHITGHRMAWDMMMNWSPTAALVVAQLLRIPQAIMDMIAGAHWGVLAGI AYFSMVGNWAKVLVVLLLFAGVDAETHVTGGSAGRTTAGLVGLLTPGAKQNIQLIN TNGSWHINSTALNCNESLNTGWLAGLFYQHKFNSSGCPERLASCRRLTDFAQGWGPI SYANGSGLDERPYCWHYPPRPCGIVPAKSVCGPVYCFTPSPVVVGTTDRSGAPTYSW GANDTDVFVLNNTRPPLGNWFGCTWMNSTGFTKVCGAPPCVIGGVGNNTLLCPTDC FRKHPEATYSRCGSGPWITPRCMVDYPYRLWHYPCTINYTIFKVRMNVSGVEHRLEA ACNWTRGERCDLEDRDRSE (SEQ ID NO:8). A Y632N-G634S substitutions is shown in bold, wherein the Y632N-G634S numbering is based on the residue positions of E2 in H77 (SEQ ID NO:1).

### C. Nucleic Acid Sequences

Disclosed are polynucleotides comprising a nucleic acid sequence capable of encoding one or more of the disclosed modified HCV glycoproteins.

### D. Vectors

Disclosed are vectors comprising any of the polynucleotides disclosed herein.

The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

In some aspects, the vector can be a viral vector. For example, the viral vector can be an adeno-associated viral vector. In some aspects, the vector can be a non-viral vector, such as a DNA based vector.

1. Viral and Non-Viral Vectors

There are a number of compositions and methods which can be used to deliver the disclosed nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Expression vectors can be any nucleotide construction used to deliver genes or gene fragments into cells (e.g., a plasmid), or as part of a general strategy to deliver genes or gene fragments, e.g., as part of recombinant retrovirus or adenovirus (Ram et al. Cancer Res. 53:83-88, (1993)). For example, disclosed herein are expression vectors comprising a nucleic acid sequence capable of encoding a VMD2 promoter operably linked to a nucleic acid sequence encoding Rapla.

The “control elements” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or pSPORT1 plasmid (Gibco BRL, Gaithersburg, Md.) and the like may be used. If it is necessary to generate a cell line that contains multiple copies of the sequence encoding a polypeptide, vectors based on SV40 or EBV may be advantageously used with an appropriate selectable marker.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Optionally, the promoter or enhancer region can act as a constitutive promoter or enhancer to maximize expression of the polynucleotides of the invention. In certain constructs the promoter or enhancer region can be active in all eukaryotic cell types, even if it is only expressed in a particular type of cell at a particular time.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases.

The expression vectors can include a nucleic acid sequence encoding a marker product. This marker product can be used to determine if the gene has been delivered to the cell and once delivered is being expressed. Marker genes can include, but are not limited to the E. coli lacZ gene, which encodes ß-galactosidase, and the gene encoding the green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

Another type of selection that can be used with the composition and methods disclosed herein is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puromycin.

As used herein, plasmid or viral vectors are agents that transport the disclosed nucleic acids, such as a nucleic acid sequence capable of encoding one or more of the disclosed peptides into the cell without degradation and include a promoter yielding expression of the gene in the cells into which it is delivered. In some embodiments the nucleic acid sequences disclosed herein are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Moloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction abilities (i.e., ability to introduce genes) than chemical or physical methods of introducing genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

Retroviral vectors, in general, are described by Verma, I. M., Retroviral vectors for gene transfer. In Microbiology, Amer. Soc. for Microbiology, pp. 229-232, Washington, (1985), which is hereby incorporated by reference in its entirety. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference in their entirety for their teaching of methods for using retroviral vectors for gene therapy.

A retrovirus is essentially a package which has packed into its nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome contains the gag, pol, and env genes which are involved in the making of the protein coat. It is the gag, pol, and env genes which are typically replaced by the foreign DNA that is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serves as the priming site for the synthesis of the second strand of DNA synthesis, and specific sequences near the ends of the LTRs that enable the insertion of the DNA state of the retrovirus to insert into the host genome. This amount of nucleic acid is sufficient for the delivery of one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)) the teachings of which are incorporated herein by reference in their entirety for their teaching of methods for using retroviral vectors for gene therapy. Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol., 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. Optionally, both the E1 and E3 genes are removed from the adenovirus genome.

Another type of viral vector that can be used to introduce the polynucleotides of the invention into a cell is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference in its entirety for material related to the AAV vector.

The inserted genes in viral and retroviral vectors usually contain promoters, or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Other useful systems include, for example, replicating and host-restricted non-replicating vaccinia virus vectors. In addition, the disclosed nucleic acid sequences can be delivered to a target cell in a non-nucleic acid based system. For example, the disclosed polynucleotides can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed expression vectors, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subject's lung cells. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

### E. Cells and Cell Lines

Disclosed herein are cells and cell lines comprising the disclosed modified hepatitis C virus (HCV) E2 glycoproteins, nucleic acid sequences, vectors or compositions disclosed herein.

As used herein, the terms “cell,” “cell line,” and “cell culture” can be used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

Suitable host cells for cloning or expressing the DNA or harboring the disclosed modified HCV E2 glycoproteins or modified membrane bound HCV E1E2 glycoproteins are the prokaryote, yeast, or higher eukaryote cells. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR(CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1.982)); MRC 5 cells; FS4 cells; and a human hepatoma line (Hep G2).

Host cells are transformed with the above-described expression or cloning vectors for modified HCV E2 glycoprotein or modified membrane bound HCV E1E2 glycoprotein production and cultured in conventional nutrient media modified as appropriate for inducing promoters, selecting transformants, or amplifying the genes encoding the desired sequences.

The modified HCV E2 glycoprotein or modified membrane bound HCV E1E2 glycoprotein composition prepared from the cells can be purified using, for example, hydroxylapatite chromatography, gel electrophoresis, dialysis, and affinity chromatography, and the like as known in the art. For example, antibodies against E2 protein can be used as affinity reagents for purification. The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

### F. Compositions

Disclosed are compositions comprising one or more of the modified HCV E2 glycoproteins or modified membrane bound HCV E1E2 glycoproteins described herein and a pharmaceutically acceptable carrier thereof.

In some aspects, the composition can be a pharmaceutical composition (e.g., formulation, preparation, medicament) comprising, or consisting essentially of, or consisting of as an active ingredient, a modified HCV E2 glycoprotein, modified membrane bound HCV E1E2 glycoprotein, a nucleic acid construct, vector, or protein as described herein, and a pharmaceutically acceptable carrier, diluent, or excipient.

Disclosed are compositions and formulations of the disclosed modified HCV E2 glycoproteins or modified membrane bound HCV E1E2 glycoproteins with a pharmaceutically acceptable carrier or diluent. For example, disclosed are pharmaceutical compositions, comprising the modified HCV E2 glycoproteins or modified membrane bound HCV E1E2 glycoproteins disclosed herein, and a pharmaceutically acceptable carrier.

For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, Pa. 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. In the methods described herein, delivery of the disclosed compositions to cells can be via a variety of mechanisms. The pharmaceutical composition may be administered in a number of ways depending on whether local or systemic treatment is desired, and on the area to be treated.

In some aspects, the disclosed compositions can be a vaccine. A vaccine is a pharmaceutical composition that is safe to administer to a subject animal, and is able to induce protective immunity in that animal against a pathogenic micro-organism, i.e. to induce a successful protection against an infection with the micro-organism. In some aspects, protection against an infection with a micro-organism is aiding in preventing, ameliorating or curing an infection with that micro-organism or a disorder arising from that infection, for example to prevent or reduce one or more clinical signs associated with the infection with the pathogen.

By the term “vaccine” as used herein, is meant a composition; a formulation comprising a composition of the invention; a virus or virus-like particle comprising a modified HCV E2 glycoprotein or modified membrane bound HCV E1E2 glycoprotein of the invention; or a nucleic acid sequence encoding a modified HCV E2 glycoprotein or modified membrane bound HCV E1E2 glycoprotein disclosed herein, which, when administered to a subject, induces cellular or humoral immune responses as described herein.

Some embodiments and compositions described herein provide a method of stimulating an immune response in a mammal, which can be a human or a preclinical model for human disease, e.g. mouse, ape, monkey etc. “Stimulating an immune response” includes, but is not limited to, inducing a therapeutic or prophylactic effect that is mediated by the immune system of the mammal. More specifically, stimulating an immune response in the context of the invention refers to eliciting cellular or humoral immune responses, thereby inducing downstream effects such as production of antibodies, antibody heavy chain class switching, maturation of APCs, and stimulation of cytolytic T cells, T helper cells and both T and B memory cells.

As appreciated by skilled artisans, vaccine compositions are suitably formulated to be compatible with the intended route of administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. The pH of the composition can be adjusted with acids or bases, such as hydrochloric acid or sodium hydroxide. Systemic administration of the composition is also suitably accomplished by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories.

Vaccine compositions may include an aqueous medium, pharmaceutically acceptable inert excipient such as lactose, starch, calcium carbonate, and sodium citrate. Vaccine compositions may also include an adjuvant, for example Freud's adjuvant. Vaccines may be administered alone or in combination with a physiologically acceptable vehicle that is suitable for administration to humans. Vaccines may be delivered orally, parenterally, intramuscularly, intranasally or intravenously. Oral delivery may encompass, for example, adding the compositions to the feed or drink of the mammals. Factors bearing on the vaccine dosage include, for example, the weight and age of the mammal. Compositions for parenteral or intravenous delivery may also include emulsifying or suspending agents or diluents to control the delivery and dose amount of the vaccine.

The modified hepatitis C virus (HCV) E2 glycoprotein and modified membrane bound HCV E1E2 glycoprotein and polynucleotides that encode such modified hepatitis C virus (HCV) E2 glycoprotein and modified membrane bound HCV E1E2 glycoproteins can be used in various HCV vaccine formulations known in the art, as a substitution for a wild-type HCV E2 sequence.

In some aspects, disclosed are vaccines comprising a modified hepatitis C virus (HCV) E2 glycoprotein comprising an antigenic domain D, wherein the modified hepatitis C virus (HCV) E2 glycoprotein comprises one or more amino acid alterations in the antigenic domain D.

In some aspects, disclosed are vaccines comprising a modified HCV E1E2 heterodimer comprising an HCV E1 glycoprotein and a modified HCV E2 glycoprotein, wherein the modified HCV E2 glycoprotein comprises an antigenic domain D, wherein the modified HCV E2 glycoproteins comprise one or more amino acid alterations in the antigenic domain D. In some aspects, at least one amino acid alteration is a proline substitution as disclosed herein.

In some aspects, disclosed are vaccines comprising a HCV E1E2 heterodimer comprising an HCV E1 glycoprotein and a modified HCV E2 glycoprotein, wherein the modified HCV E2 glycoprotein is a membrane bound E2 glycoprotein comprising an antigenic domain D, wherein the modified HCV E2 glycoproteins comprise one or more amino acid alterations in the antigenic domain D. In some aspects, at least one amino acid alteration is a proline substitution as disclosed herein.

In some aspects, disclosed are vaccines comprising a modified HCV E2 glycoprotein comprising an antigenic domain D, wherein the modified HCV E2 glycoproteins comprise one or more amino acid alterations in the antigenic domain D, wherein at least one amino acid alteration is a proline substitution. For example, disclosed are vaccines comprising a modified HCV E2 glycoprotein comprising the sequence of SEQ ID NO:2.

The disclosed modified HCV E2 glycoproteins or modified membrane bound HCV E1E2 glycoproteins and nucleic acid sequences that encode such modified HCV E2 glycoproteins or modified membrane bound HCV E1E2 glycoproteins can be used in various HCV vaccine formulations known in the art, as a substitution for the wild-type HCV E2 sequence. In some aspects, the disclosed vaccines are live-attenuated virus, replication-defective viruses, nanoparticles, or subunit vaccines wherein each of them comprise one of the disclosed modified HCV E2 glycoproteins or modified membrane bound HCV E1E2 glycoproteins. In some aspects, the modified HCV E2 glycoproteins or modified membrane bound HCV E1E2 glycoproteins can help form a live-attenuated virus or replication-defective virus vaccine. In some aspects, the disclosed vaccines can be mRNA vaccines comprising one of the disclosed nucleic acid sequences. For example, the disclosed vaccines can be mRNA vaccines comprising a nucleic acid sequence that encodes one of the disclosed modified HCV E2 glycoproteins or modified membrane bound HCV E1E2 glycoproteins.

1. Delivery of Compositions

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

### G. Methods

Disclosed are methods of increasing HCV E2 glycoprotein antigenicity in a subject in need thereof comprising administering a composition comprising one or more of the modified HCV E2 glycoproteins described herein, wherein the increase in HCV E2 glycoprotein antigenicity is an increase in antigenic domain D antigenicity. In some aspects, disclosed are methods of increasing HCV E2 glycoprotein antigenicity in a subject in need thereof comprising administering a composition comprising one or more of the modified HCV E2 glycoproteins disclosed herein, wherein the modified HCV E2 glycoprotein comprises an antigenic domain D, wherein the modified HCV E2 glycoproteins comprise one or more amino acid alterations in the antigenic domain D, wherein at least one amino acid alteration is a proline substitution, and wherein the increase in HCV E2 glycoprotein antigenicity is an increase in antigenic domain D antigenicity. For example, a proline substitutions can be a proline substitution as disclosed herein, such as the H445P substitution found in SEQ ID NO:2 and can increase the antigenicity of HCV E2 glycoprotein. In some aspects, the presence of a proline substitution in the antigenic domain D near an antibody binding site can help stabilize the epitope resulting in increased antigenicity. In some aspects, the modified HCV E2 glycoprotein can further comprise an N-glycan sequon in the antigenic domain A. In some aspects, the modified HCV E2 glycoprotein can further comprise an N-glycan sequon in the antigenic domain A wherein the antigenicity of antigenic domain A is masked and the antigenicity in antigenic domain D is increased.

Disclosed are methods of decreasing HCV E2 glycoprotein antigenicity in a subject in need thereof comprising administering a composition comprising one or more of the modified HCV E2 glycoproteins described herein, wherein the decrease in HCV E2 glycoprotein antigenicity is a decrease in antigenic domain A antigenicity. In some aspects, disclosed are method of decreasing HCV E2 glycoprotein antigenicity in a subject in need thereof comprising administering a composition comprising one or more of the modified HCV E2 glycoproteins comprising an antigenic domain A, wherein the antigenic domain A comprises an N-glycan sequon substitution, wherein the decrease in HCV E2 glycoprotein antigenicity is a decrease in antigenic domain A antigenicity. In some aspects, the N-glycan sequeon substitution in the antigenic domain A masks an epitope, therefore decreasing the antigenicity of antigenic domain A. In some aspects, the antigenic domain A is known to be associated with non-neutralizing antibodies. In some aspects, by masking this region and diverting the antibody response to other regions, such as the antigenic domain D, that neutralizing antibodies can bind can be a good mechanism for vaccine development. In some aspects, any of the modified HCV E2 glycoproteins comprising the N-glycan sequon substitution in the antigenic domain A can be used in these methods.

Disclosed are methods of inducing an immune response in a subject in need thereof comprising administering to the subject in need thereof a composition comprising one or more of the modified HCV E2 glycoproteins disclosed herein. Disclosed are methods of inducing an immune response in a subject in need thereof comprising administering to the subject in need thereof a composition comprising one or more of the modified HCV E2 glycoproteins comprising an antigenic domain D, wherein the modified HCV E2 glycoproteins comprise one or more amino acid alterations in the antigenic domain D, wherein at least one amino acid alteration is a proline substitution. In some aspects of the disclosed methods of inducing an immune response in a subject in need thereof, the immune response is an antibody response wherein the antibodies can bind to HCV. In some aspects, the modified HCV E2 glycoproteins comprising an antigenic domain D, wherein the modified HCV E2 glycoproteins comprise one or more amino acid alterations in the antigenic domain D, wherein at least one amino acid alteration is a proline substitution induces a stronger or more potent antibody response than an HCV E2 glycoprotein not having a proline substitution in the antigenic domain D. For example, the modified HCV E2 glycoproteins comprising an antigenic domain D, wherein the modified HCV E2 glycoproteins comprise one or more amino acid alterations in the antigenic domain D, wherein at least one amino acid alteration is a proline substitution induces a stronger or more potent antibody response than the wild type H77 E2 glycoprotein.

In some aspects of any of the disclosed methods herein, the subject in need thereof has been infected with hepatitis C virus (HCV) or is at risk for being infected with HCV.

Also disclosed are methods of treating a subject having HCV or at risk of being infected with HCV comprising administering to the subject a composition comprising one or more of the modified HCV E2 glycoproteins disclosed herein. In some aspects, treating a subject can include preventing further infection in a subject already infected with HCV. In some aspects, treating a subject can include preventing infection or viral replication in a subject exposed to HCV. In some aspects, the modified HCV E2 glycoprotein induces an immune response against HCV in the subjects. In some aspects, the modified HCV E2 glycoproteins can be any of the modified HCV E2 glycoproteins comprising a proline substitution in the antigenic domain D. In some aspects, the modified HCV E2 glycoproteins comprising a proline substitution in the antigenic domain D and an N-glycan sequon substitution in antigenic domain A.

Disclosed are methods of generating neutralizing antibodies (nAbs) to the antigenic domain D of HCV in a subject in need thereof comprising administering to the subject in need thereof a composition comprising one or more of the modified HCV E2 glycoproteins described herein. In some aspects, the subject in need thereof has been infected with HCV or is at risk for being infected with HCV. In some aspects, the modified HCV E2 glycoproteins can be any of the modified HCV E2 glycoproteins comprising a proline substitution in the antigenic domain D. In some aspects, the modified HCV E2 glycoproteins comprising a proline substitution in the antigenic domain D and an N-glycan sequon substitution in antigenic domain A.

Also disclosed are methods for immunizing a subject in need thereof comprising administering to the subject in need thereof a composition comprising one or more of the modified HCV E2 glycoproteins disclosed herein. In some aspects, the subject in need thereof has been infected with HCV or is at risk for being infected with HCV. In some aspects, the modified HCV E2 glycoproteins can be any of the modified HCV E2 glycoproteins comprising a proline substitution in the antigenic domain D. In some aspects, the modified HCV E2 glycoproteins comprising a proline substitution in the antigenic domain D and an N-glycan sequon substitution in antigenic domain A. In some aspects, a protective immune response effective to reduce or eliminate subsequent HCV infection clinical signs in the subject, relative to a non-immunized control subject of the same species, is elicited by administration of the composition. In some aspects, a protective immune response effective to reduce risk of HCV infection in the subject, relative to a non-immunized control subject of the same species, is elicited by administration of the composition.

In the methods disclosed herein, an immunologically effective amount of one or more disclosed modified HCV E2 glycoproteins or modified membrane bound HCV E1E2 glycoproteins, which may be conjugated to a suitable carrier molecule, polynucleotides encoding such modified polypeptides, including viral vectors, are administered to a subject by administrations of a vaccine, in a manner effective to result in an improvement in the subject's condition.

In some aspects of any of the disclosed methods, the composition can be administered in a therapeutically effective amount. By an “effective amount” of a composition as provided herein is meant a sufficient amount of the composition to provide the desired effect. The exact amount required will vary from subject to subject, depending on the species, age, and general condition of the subject, the severity of disease (or underlying genetic defect) that is being treated, the particular composition used, its mode of administration, and the like. Thus, it is not possible to specify an exact “effective amount.” However, an appropriate “effective amount” may be determined by one of skill in the art using only routine experimentation. The term “therapeutically effective amount” means an amount of a therapeutic, prophylactic, and/or diagnostic agent (e.g., modified HCV E2 glycoprotein) that is sufficient, when administered to a subject suffering from or susceptible to infection with HCV, to treat, alleviate, ameliorate, relieve, alleviate symptoms of, prevent, delay onset of, inhibit progression of, reduce severity of, and/or reduce incidence of infection with HCV. The term “immunologically effective amount” means an amount of a therapeutic, prophylactic, and/or diagnostic agent (e.g., modified HCV E2 glycoproteins or modified membrane bound HCV E1E2 glycoproteins) that is sufficient, when administered to a subject suffering from or susceptible to infection with HCV, to treat, alleviate, ameliorate, relieve, alleviate symptoms of, prevent, delay onset of, inhibit progression of, reduce severity of, and/or reduce incidence of infection with HCV based on an immune response.

In some aspects, the modified glycoproteins are used in a screening method to select for antibodies optimized for affinity, specificity, and the like. In such screening methods, random or directed mutagenesis is utilized to generate changes in the amino acid structure of the variable region or regions, where such variable regions will initially comprise one or more of the provided CDR sequences, e.g. a framework variable region comprising CDR1, CDR2, CDR3 from the heavy and light chain sequences. Methods for selection of antibodies with optimized specificity, affinity, etc., are known and practiced in the art, e.g. including methods described by Presta (2006) Adv Drug Deliv Rev. 58(5-6):640-56; Levin and Weiss (2006) Mol Biosyst. 2(1):49-57; Rothe et al. (2006) Expert Opin Biol Ther. 6(2):177-87; Ladner et al. (2001) Curr Opin Biotechnol. 12(4):406-10; Amstutz et al. (2001) Curr Opin Biotechnol. 12(4):400-5; Nakamura and Takeo (1998) J Chromatogr B Biomed Sci Appl. 715(1):125-36 each herein specifically incorporated by reference for teaching methods of mutagenesis selection. Such methods are exemplified by Wu et al. (2005) J. Mol. Biol. (2005) 350, 126-144.

In some aspects, any of the disclosed methods can be performed by administering one or more of the disclosed modified membrane bound HCV E1E2 glycoproteins instead of or in addition to the disclosed modified HCV E2 glycoproteins.

Disclosed are methods of increasing HCV E2 glycoprotein antigenicity in a subject in need thereof comprising administering a composition comprising one or more of the modified membrane bound HCV E1E2 glycoproteins described herein, wherein the increase in HCV E2 glycoprotein antigenicity is an increase in antigenic domain D antigenicity. In some aspects, disclosed are methods of increasing HCV E2 glycoprotein antigenicity in a subject in need thereof comprising administering a composition comprising one or more of the modified membrane bound HCV E1E2 glycoproteins disclosed herein, wherein the modified HCV E2 glycoprotein comprises an antigenic domain D, wherein the modified membrane bound HCV E1E2 glycoproteins comprise one or more amino acid alterations in the antigenic domain D, wherein at least one amino acid alteration is a proline substitution, and wherein the increase in HCV E2 glycoprotein antigenicity is an increase in antigenic domain D antigenicity. For example, a proline substitutions can be a proline substitution as disclosed herein, such as the H445P substitution found in SEQ ID NO: 2 and can increase the antigenicity of HCV E2 glycoprotein. In some aspects, the presence of a proline substitution in the antigenic domain D near an antibody binding site can help stabilize the epitope resulting in increased antigenicity. In some aspects, the modified membrane bound HCV E1E2 glycoprotein can further comprise an N-glycan sequon in the antigenic domain A. In some aspects, the modified membrane bound HCV E1E2 glycoprotein can further comprise an N-glycan sequon in the antigenic domain A wherein the antigenicity of antigenic domain A is masked and the antigenicity in antigenic domain D is increased.

Disclosed are method of decreasing HCV E2 glycoprotein antigenicity in a subject in need thereof comprising administering a composition comprising one or more of the modified membrane bound HCV E1E2 glycoproteins described herein, wherein the decrease in HCV E2 glycoprotein antigenicity is a decrease in antigenic domain A antigenicity. In some aspects, disclosed are method of decreasing HCV E2 glycoprotein antigenicity in a subject in need thereof comprising administering a composition comprising one or more of the modified membrane bound HCV E1E2 glycoproteins comprising an antigenic domain A, wherein the antigenic domain A comprises an N-glycan sequon substitution, wherein the decrease in HCV E2 glycoprotein antigenicity is a decrease in antigenic domain A antigenicity. In some aspects, the N-glycan sequeon substitution in the antigenic domain A masks an epitope, therefore decreasing the antigenicity of antigenic domain A. In some aspects, the antigenic domain A is known to be associated with non-neutralizing antibodies. In some aspects, by masking this region and diverting the antibody response to other regions, such as the antigenic domain D, that neutralizing antibodies can bind can be a good mechanism for vaccine development. In some aspects, any of the modified membrane bound HCV E1E2 glycoproteins comprising the N-glycan sequon substitution in the antigenic domain A can be used in these methods.

Disclosed are methods of inducing an immune response in a subject in need thereof comprising administering to the subject in need thereof a composition comprising one or more of the modified membrane bound HCV E1E2 glycoproteins disclosed herein. Disclosed are methods of inducing an immune response in a subject in need thereof comprising administering to the subject in need thereof a composition comprising one or more of the modified membrane bound HCV E1E2 glycoproteins comprising an antigenic domain D, wherein the modified HCV E2 glycoproteins comprise one or more amino acid alterations in the antigenic domain D, wherein at least one amino acid alteration is a proline substitution. In some aspects of the disclosed methods of inducing an immune response in a subject in need thereof, the immune response is an antibody response wherein the antibodies can bind to HCV. In some aspects, the modified membrane bound HCV E1E2 glycoproteins comprising an antigenic domain D, wherein the membrane bound HCV E1E2 heterodimers comprise one or more amino acid alterations in the antigenic domain D, wherein at least one amino acid alteration is a proline substitution induces a stronger or more potent antibody response than a modified membrane bound HCV E1E2 glycoprotein not having a proline substitution in the antigenic domain D. For example, the modified membrane bound HCV E1E2 glycoproteins comprising an antigenic domain D, wherein the modified membrane bound HCV E1E2 glycoproteins comprise one or more amino acid alterations in the antigenic domain D, wherein at least one amino acid alteration is a proline substitution induces a stronger or more potent antibody response than the wild type H77 modified membrane bound HCV E1E2 glycoproteins.

In some aspects of any of the disclosed methods herein, the subject in need thereof has been infected with hepatitis C virus (HCV) or is at risk for being infected with HCV.

Also disclosed are methods of treating a subject having HCV or at risk of being infected with HCV comprising administering to the subject a composition comprising one or more of the modified membrane bound HCV E1E2 glycoproteins disclosed herein. In some aspects, treating a subject can include preventing further infection in a subject already infected with HCV. In some aspects, treating a subject can include preventing infection or viral replication in a subject exposed to HCV. In some aspects, the modified membrane bound HCV E1E2 glycoprotein induces an immune response against HCV in the subjects. In some aspects, the modified membrane bound HCV E1E2 glycoproteins can be any of the membrane bound HCV E1E2 glycoproteins comprising a proline substitution in the antigenic domain D. In some aspects, the modified membrane bound HCV E1E2 glycoproteins comprising a proline substitution in the antigenic domain D and an N-glycan sequon substitution in antigenic domain A.

Disclosed are methods of generating neutralizing antibodies (nAbs) to the antigenic domain D of HCV in a subject in need thereof comprising administering to the subject in need thereof a composition comprising one or more of the modified membrane bound HCV E1E2 glycoproteins described herein. In some aspects, the subject in need thereof has been infected with HCV or is at risk for being infected with HCV. In some aspects, the modified membrane bound HCV E1E2 glycoproteins can be any of the modified membrane bound HCV E1E2 glycoproteins comprising a proline substitution in the antigenic domain D. In some aspects, the modified membrane bound HCV E1E2 glycoproteins comprising a proline substitution in the antigenic domain D and an N-glycan sequon substitution in antigenic domain A.

Also disclosed are methods for immunizing a subject in need thereof comprising administering to the subject in need thereof a composition comprising one or more of the modified membrane bound HCV E1E2 glycoproteins disclosed herein. In some aspects, the subject in need thereof has been infected with HCV or is at risk for being infected with HCV. In some aspects, the modified membrane bound HCV E1E2 heterodimers can be any of the modified membrane bound HCV E1E2 heterodimers comprising a proline substitution in the antigenic domain D. In some aspects, the modified membrane bound HCV E1E2 glycoproteins comprising a proline substitution in the antigenic domain D and an N-glycan sequon substitution in antigenic domain A. In some aspects, a protective immune response effective to reduce or eliminate subsequent HCV infection clinical signs in the subject, relative to a non-immunized control subject of the same species, is elicited by administration of the composition. In some aspects, a protective immune response effective to reduce risk of HCV infection in the subject, relative to a non-immunized control subject of the same species, is elicited by administration of the composition.

In some aspects of any of the disclosed methods, the composition can be administered in a therapeutically effective amount. By an “effective amount” of a composition as provided herein is meant a sufficient amount of the composition to provide the desired effect. The exact amount required will vary from subject to subject, depending on the species, age, and general condition of the subject, the severity of disease (or underlying genetic defect) that is being treated, the particular composition used, its mode of administration, and the like. Thus, it is not possible to specify an exact “effective amount.” However, an appropriate “effective amount” may be determined by one of ordinary skill in the art using only routine experimentation. The term “therapeutically effective amount” means an amount of a therapeutic, prophylactic, and/or diagnostic agent (e.g., modified HCV E2 glycoprotein) that is sufficient, when administered to a subject suffering from or susceptible to infection with HCV, to treat, alleviate, ameliorate, relieve, alleviate symptoms of, prevent, delay onset of, inhibit progression of, reduce severity of, and/or reduce incidence of infection with HCV.

In some aspects of the disclosed methods, the composition can be administered subcutaneously, intramuscularly, intravenously, intradermally, or orally.

### H. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of the disclosed glycoproteins, nucleic acids, vectors, or compositions.

### Examples

### 1. Introduction

Described herein is the generation, characterization, and in vivo immunogenicity of structure-based designs of the HCV E2 glycoprotein, which is the primary target of the antibody response to HCV and a major vaccine target. Designs were focused on antigenic domain D, which is a key region of E2 targeted by broadly neutralizing antibodies (bNAbs) that are resistant to viral escape, as well as antigenic domain A, which is targeted by non-neutralizing antibodies. Based on the intrinsic flexibility of the neutralizing face of E2, which includes antigenic domain D, and on the locations of bNAb epitopes to this domain, a structure-based design substitution was identified to reduce the mobility of that region and preferentially form the bnAb-bound conformation. Several substitutions were also tested to hyperglycosylate and mask antigenic domain A located in a unique region on the back layer of E2, as determined by fine epitope mapping, which represents an approach that has been applied to mask epitopes in influenza and HIV glycoproteins. Designs were tested for antigenicity using a panel of monoclonal antibodies (mAbs), and selected designs were tested individually and as combinations for in vivo immunogenicity. Assessment of immunized sera revealed that certain E2 designs yielded improvements in serum binding to recombinant HCV particles, as well as viral cross-neutralization, while maintaining serum binding to soluble E2 glycoprotein and key epitopes. Rational design of HCV glycoproteins can lead to improvements in immunogenicity and neutralization breadth.

### 2. Results

i. Structure-Based Design of E2

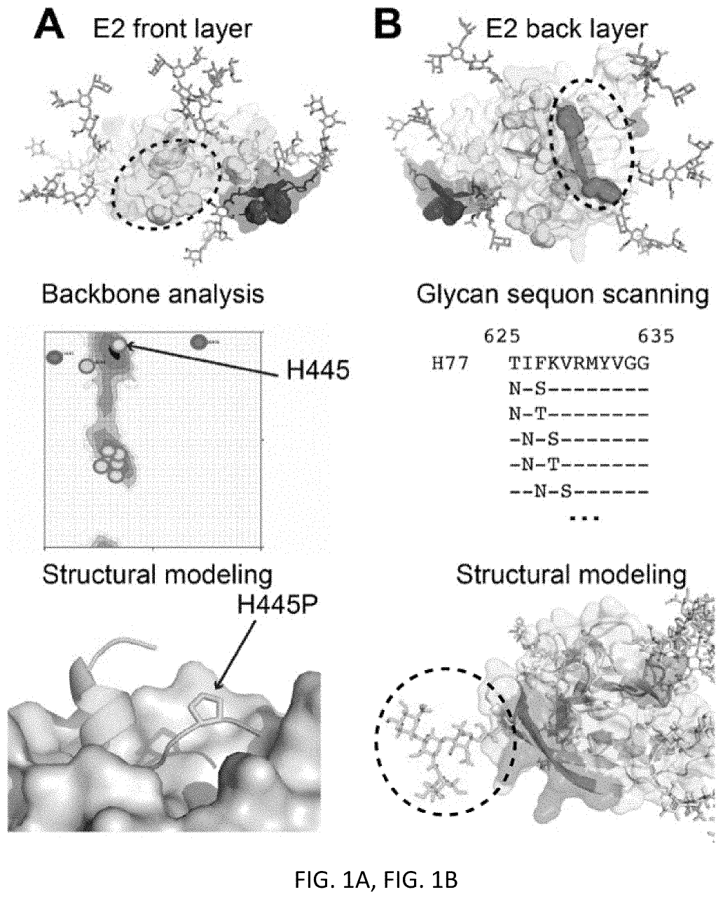

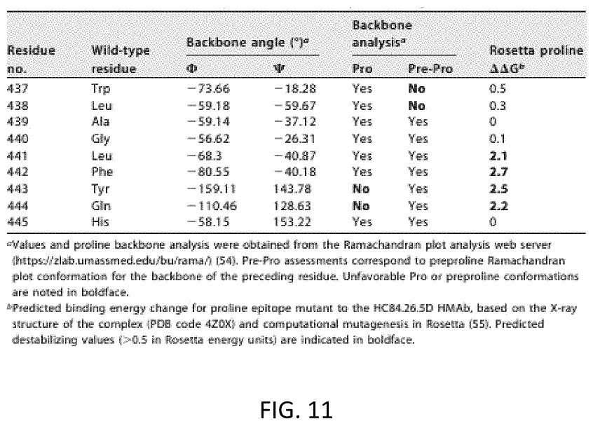

Two approaches were utilized to design variants of the E2 glycoprotein to improve its antigenicity and immunogenicity (FIG. 1). For one approach, the previously reported structure of the affinity matured bnAb HC84.26.5D bound to its epitope from E2 antigenic domain D was used (PDB code 4Z0X), which shows the same epitope conformation observed in the context of other domain D human monoclonal antibodies (HMAbs) targeting this site. Analysis of this epitope structure for potential proline residue substitutions to stabilize its HMAb-bound conformation identified several candidate sites (FIG. 1A, FIG. 11). One of these substitutions, H445P, that is adjacent to core contact residues for domain D located at aa 442-443 was selected for subsequent experimental characterization, due to its position in a region with no secondary structure, and location between residues Y443 and K446 which both make key antibody contacts in domain D antibody complex structures. This also represents a distinct region of the epitope from the substitution previously described and tested, A439P.

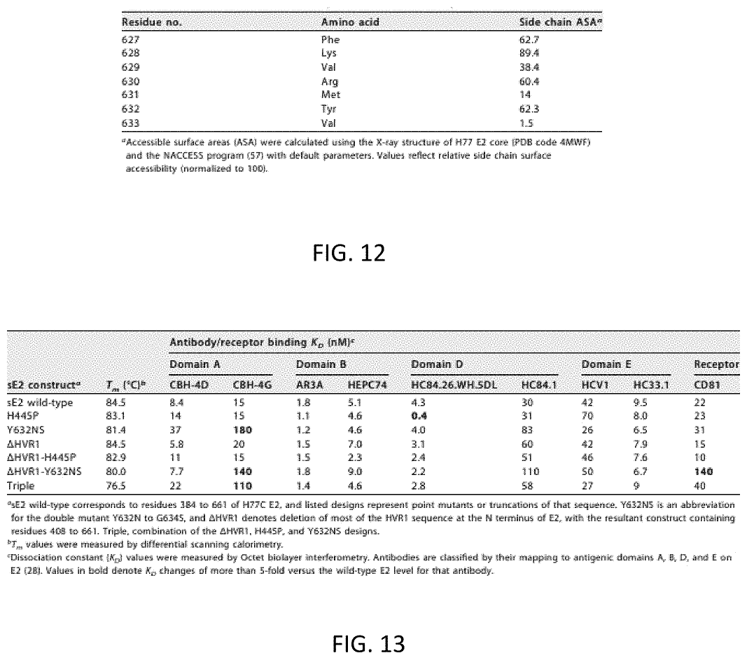

Another design approach, hyperglycosylation, was utilized to mask antigenic domain A, which is an immunogenic region on the back layer of E2 associated with non-neutralizing antibodies. Other antibodies with some binding determinants mapped to this region, including HMAbs ARIA and HEPC46, exhibit limited or weak neutralization. N×S (Asparagine-X-Serine) and N×T (Asparagine-X-Threonine) N-glycan sequon substitutions were modeled in Rosetta at solvent-exposed E2 positions in antigenic domain A (FIG. 1B, FIG. 12), followed by visual inspection of the modeled E2 mutant structures to confirm exposure of the mutant asparagine residues. This analysis indicated that designs with N-glycans at residues 627 (F627N-V629T), 628 (K628N-R630S), 630 (R630N-Y632T), and 632 (Y632N-G634S) were further investigated for effects on antigenicity.

ii. Initial Screening of Mutant Antigenicity Using ELISA

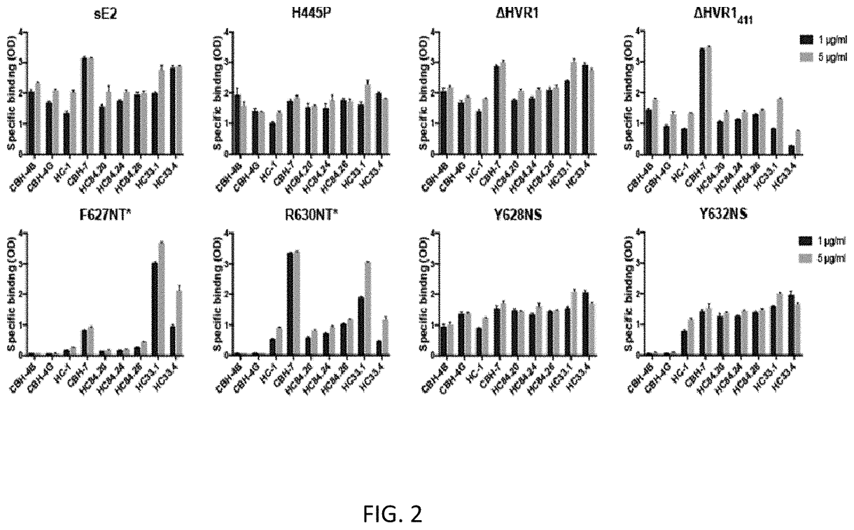

The structure-based designs described above were first screened to assess their effects on E2 glycoprotein antigenicity, to confirm that designs preserved the structure of key E2 epitopes, and to disrupt non-neutralizing antigenic domain A HMAb binding in the case of the N-glycan designs. These designs were cloned in E1E2 and assessed using ELISA with a panel of representative HMAbs to antigenic domains A-E (FIG. 2). Only two HMAb concentrations were tested in this assay, in order to detect major disruptions to HMAb binding, or lack thereof, rather than quantitative measurements. The results indicated that mutant H445P maintained approximately wild-type levels of binding to antibodies, while truncations of HVR1 had varying effects. Binding of domain E HMAb HC33.4, and to a lesser extent HC33.1, was negatively affected by truncation of all of HVR1 (residues 384-410 removed; referred to here as ΔHVR1₄₁₁), whereas a more limited HVR1 truncation (residues 384-407 removed; referred to here as ΔHVR1) largely restored binding of these bNAbs. The design of ΔHVR1 was based on the observation that residue 408 located within HVR1 affected the binding of HC33.4 but not HC33.1. Likewise, designed N-glycan substitutions showed varying effects on antigenicity, with pronounced reduction of binding for several bNAbs for F627NT (F627N-V629T) and R630NT (R630N-Y632T), while K628NS (K628N-R630S) did not exhibit ablation of domain A antibody binding. In contrast, Y632NS (Y632N-G634S) disrupted binding for both tested domain A HMAbs, with limited loss of binding for other HMAbs. Based on this antigenic characterization, designs H445P, ΔHVR1, and Y632NS were selected for further testing.

iii. Biophysical and Antigenic Characterization of E2 Designs

The two candidate structure-based E2 designs H445P, Y632NS, as well as ΔHVR1, were expressed and purified as monomeric soluble E2 (sE2) glycoproteins and tested for thermostability and binding affinity to a panel of HMAbs, as well as the CD81 receptor (FIG. 13). Pairwise combinations of these designs, and a “Triple” design with all three modifications, were also expressed and tested. As noted previously by others, wild-type sE2 was found to exhibit high thermostability (Tₘ=84.5° C. in FIG. 13). All designs likewise showed high thermostability, with only minor reductions in Tₘ, with the exception of combined Triple which had the lowest measured thermostability among the tested E2 mutants (Tₘ=76.5° C.).

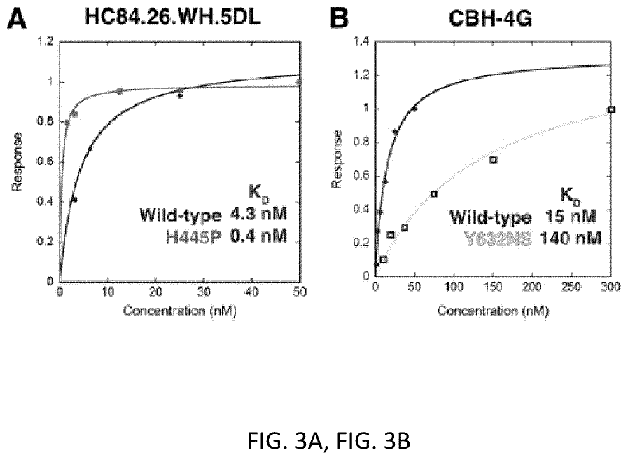

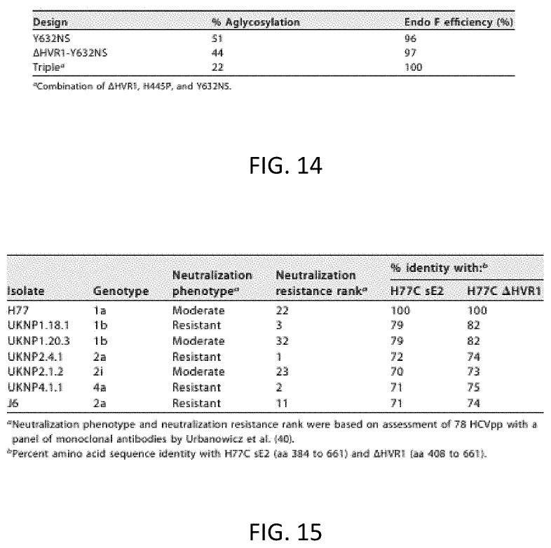

To assess antigenicity of glycoprotein designs, solution binding affinity measurements were performed with Octet using HMAbs that target E2 antigenic domains A, B, D, and E, with two antibodies per domain, as well as the receptor CD81 (FIG. 13). These antibodies have been characterized using multiple global alanine scanning studies (CBH-4G, CBH-4D, HC33.1, AR3A, HC33.1), and X-ray structural characterization studies (AR3A, HEPC74, HC84.1, HC33.1, HCV1). The HC84.26.WH.5DL is an affinity matured clone of the parental HC84.26 antibody with improved affinity and neutralization breadth over the parental antibody. The binding site of CD81 has been mapped to E2 residues in antigenic domains B, D, and E, thus CD81 binding provides additional assessment of antigenicity of that E2 supersite. Binding experiments with this panel showed nanomolar binding affinities to wild-type sE2, which were largely maintained for sE2 designs. A 10-fold increase in binding affinity of sE2 design H445P for domain D HMAb HC84.26.WH.5DL was observed, showing that this design, located within antigenic domain D, not only maintained affinity, but improved engagement in that case; a steady-state binding fit for that interaction is shown in FIG. 3A. However, this effect was not observed for combinations of designs including H445P, indicating possible interplay between designed sites. As expected, domain A hyperglycosylation designs Y632NS, ΔHVR1-Y632NS, and Triple (ΔHVR1-H445P-Y632NS) showed loss of binding (>5-fold for each) to antigenic domain A HMAb CBH-4G (Y632NS-CBH-4G binding measurement is shown in FIG. 3B), though disruption of binding to CBH-4D was not observed. Additionally, design ΔHVR1-Y632NS showed moderate (6-fold) loss of CD81 binding, which was not the case for other designs. As domain A HMAbs have distinct, albeit similar, binding determinants on E2, differential effects on domain A antibody binding by Y632NS variants reflect likely differences in HMAb docking footprints on E2. Measurements of glycan occupancy at residue 632 using mass spectroscopy showed partial levels of glycosylation at that site for Y632NS and combinations (FIG. 14), which can be responsible for incomplete binding ablation to the tested antigenic domain A HMAbs. It is possible that the Y632N amino acid substitution in the Y632NS may be responsible, in addition to partial N-glycosylation, for effects on domain A antibody binding. These results indicate at least partial binding disruption and N-glycan masking of this region, supporting testing of those designs as immunogens in vivo.

iv. In Vivo Immunogenicity of E2 Designs

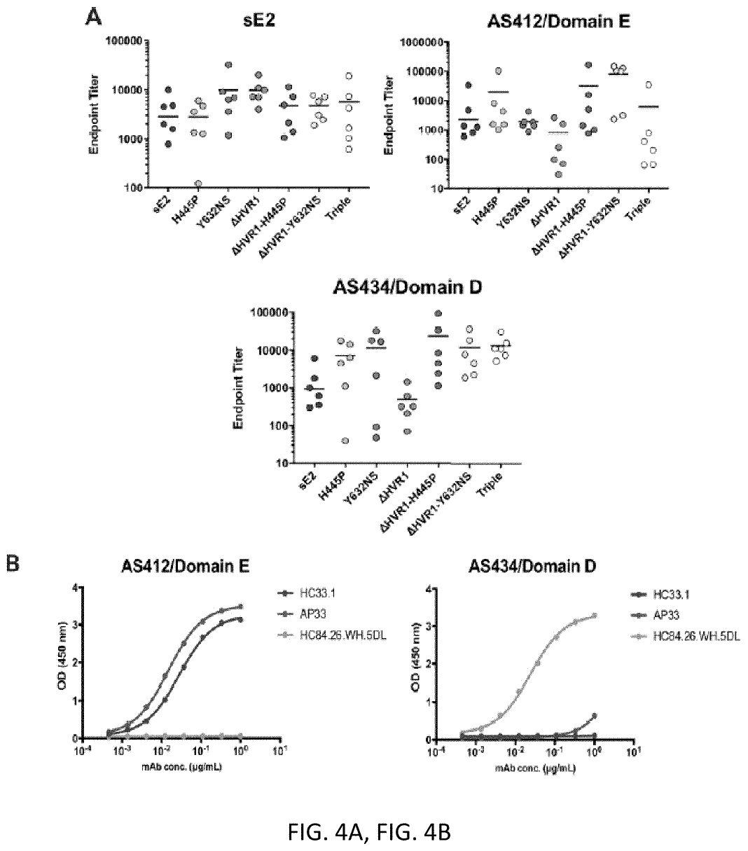

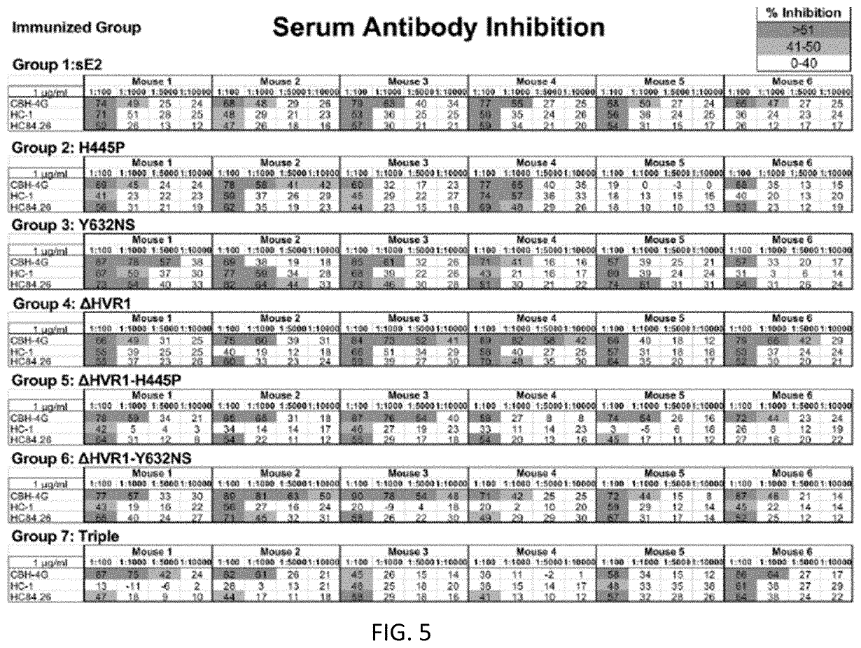

Following confirmation of antigenicity, E2 designs were tested in vivo for immunogenicity, to assess elicitation of antibodies that demonstrate potency and neutralization breadth. CD1 mice (6 per group) were immunized with H77C sE2 and designs, employing Day 0 prime followed by three biweekly boosts. Sera were obtained at Day 56 (two weeks after the final boost) and tested for binding to H77C sE2 and key conserved epitopes (AS412/Domain E, AS434/Domain D) (FIG. 4). Peptide epitopes were confirmed for expected monoclonal antibody specificity using ELISA (FIG. 4B). Endpoint titers demonstrated that sera from mice immunized with E2 designs maintained recognition of sE2 and tested epitopes. Intra-group variability resulted in lack of statistically significant differences in serum binding between immunized groups, however mean titers from ΔHVR1 group were moderately lower than the wild-type sE2 group, and other mutants yielded moderately higher serum binding to the tested epitopes. Notably, design H445P elicited antibodies that robustly cross-reacted with the wild type AS434/Domain D epitope. To assess differential binding to conformational epitopes on E2, serum binding competition with selected HMAbs was performed (FIG. 5). The observation of competition in the majority of antisera suggests that elicited antibodies to domain D are to native conformational epitopes, although there were no major differences between immunized groups. Likewise, no substantial differences in serum competition for binding to antigenic domains A or B were detected among immunized groups.

v. Serum Binding to HCV E1E2 and HCV Pseudoparticles

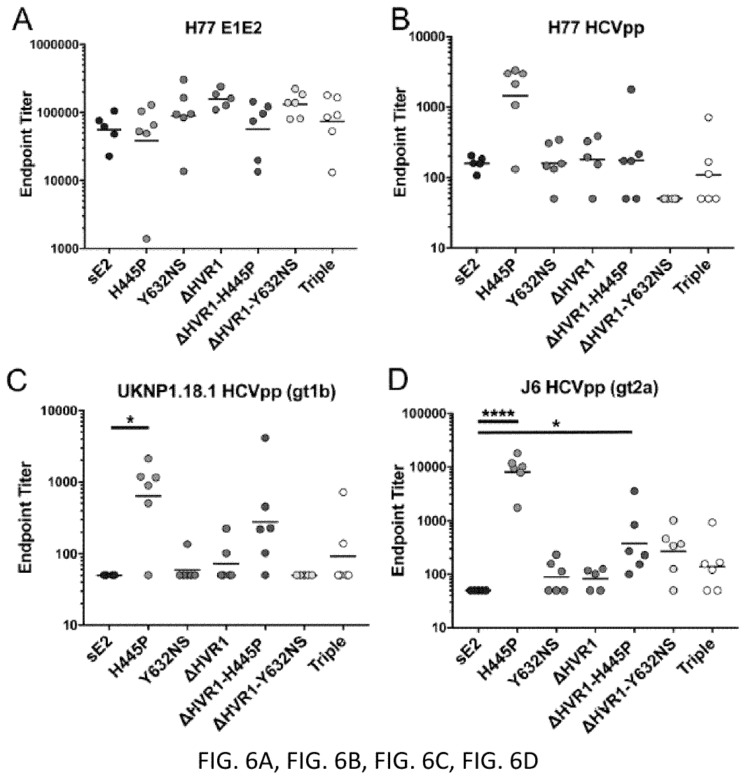

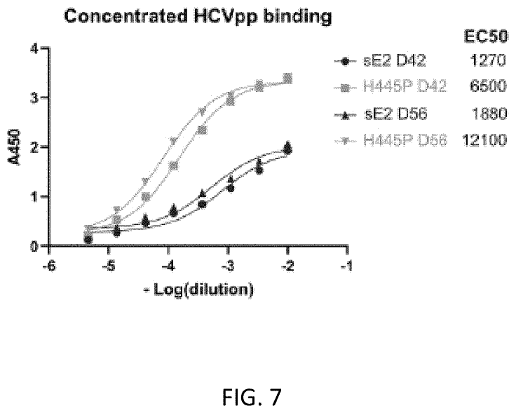

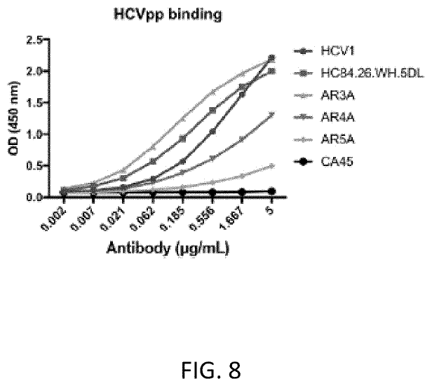

For further analysis of immunized serum binding, binding to concentrated recombinant H77C E1E2 and HCV pseudoparticles from H77C and two heterologous genotypes was assessed (FIG. 6, FIG. 15). While binding to H77 E1E2 resembled binding to H77 sE2, with no apparent difference between immunized groups, notable differences were observed in binding to HCVpps representing H77C, UKNP1.18.1, and J6 for H445P-immunized mice versus mice immunized with wild-type sE2. The difference between J6 HCVpp binding from H445P-immunized mice versus sE2-immunized mice was highly significant (p≤0.0001, Kruskal-Wallis test). To confirm this difference in HCVpp binding between sE2 and H445P immunized groups, given the relatively low levels of overall titers, H77C HCVpps were concentrated and tested in ELISA for binding to pooled sera from sE2 and H445P immunized mice. This confirmed differences between immunized groups for sera from Day 56, as well as Day 42, which corresponds to three rather than four immunizations (FIG. 7). To demonstrate native-like E2 and E1E2 assembly of the HCVpps in the context of the ELISA assay, purified HCVpps showed binding to monoclonal antibodies that target linear and conformational epitopes on E2 (HCV1, HC84.26.WH.5DL, AR3A) and conformational epitopes on E1E2 (AR4A, AR5A), and did not interact with negative control antibody (CA45) (FIG. 8). The molecular basis for the differential serum reactivity when using HCVpp versus purified recombinant E1E2 in ELISA is unclear, particularly given that sE2 was used as an immunogen, yet these results collectively provide evidence that H445P can improve targeting of conserved glycoprotein epitopes on the intact HCV virion.

vi. Homologous and Heterologous Serum Neutralization

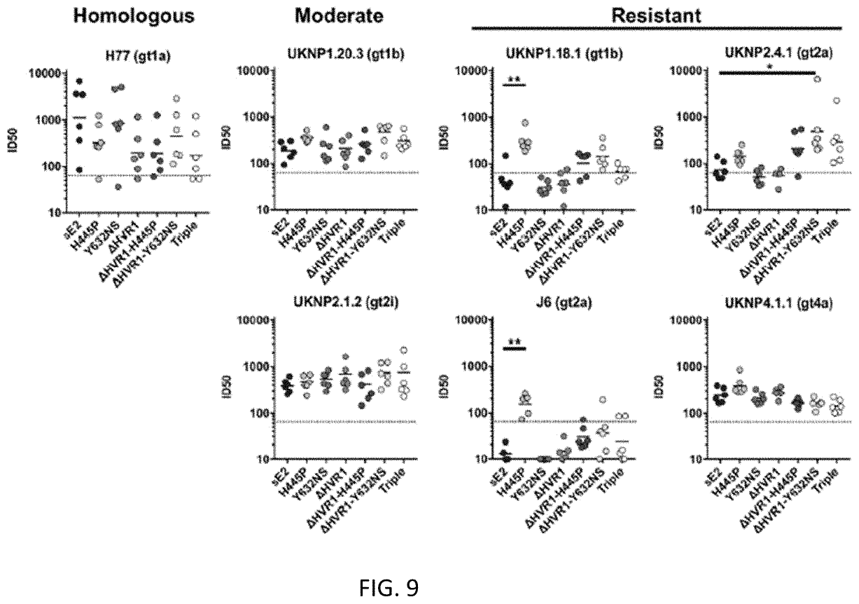

To assess effects of antibody neutralization potency and breadth from E2 designs, we tested serum neutralization of HCVpp representing homologous H77C and six heterologous isolates (FIG. 9). The heterologous isolates collectively diverge substantially in sequence from H77C and represent neutralization phenotypes ranging from moderately to highly resistant (FIG. 15), with the latter represented by three of the most resistant tested HCVpp from a previous study that performed characterization with a panel of neutralizing monoclonal antibodies (UKNP2.4.1, UKNP4.1.1, UKNP1.18.1). There was relatively large intra-group variability in neutralization of H77C, and no statistically significant differences between groups were observed. However, ID50 values for individual mice varied less within immunized groups for heterologous isolates. Comparison between groups immunized with sE2 designs and wild-type sE2 showed significantly higher neutralization in some cases. Notably, two resistant isolates had significantly higher neutralization for H445P-immunized sera than wild-type sE2-immunized sera (UKNP1.18.1, J6).

vii. Analysis of Correlates of Immunogenicity and Antigenicity

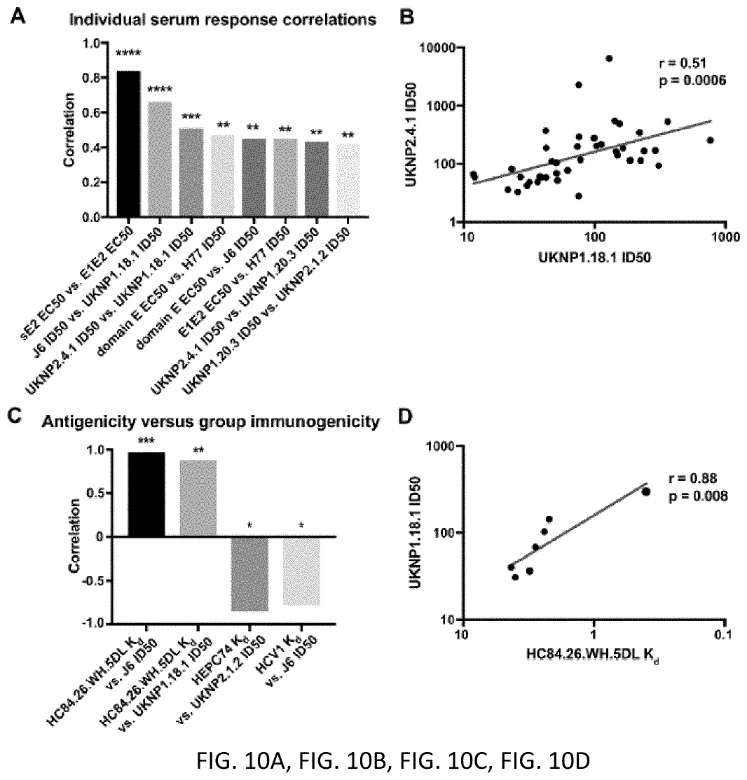

Based on our in vitro and in vivo measurements, we assessed correlations between serum neutralization of different genotypes, serum antigen binding, and antigenicity (FIG. 10). First, correlations were performed between immunogenicity measurements for individual murine sera, corresponding to 42 points per dataset. Measurements of HCVpp serum binding were not included in this analysis, due to low and unquantifiable binding measurements for multiple mice for those assays (FIG. 6B-D). Top correlations between immunogenicity measurements (FIG. 10A) include serum binding values (EC50) to sE2 versus E1E2 (r=0.84), J6 neutralization (ID50) versus UKNP1.18.1 neutralization (r=0.66), and UKNP2.4.1 neutralization versus UKNP1.18.1 neutralization (r=0.51), all of which were highly significant (p≤0.001). The latter two correlations highlight shared patterns of neutralization of HCVpp with resistant phenotypes; a plot of UKNP2.4.1 HCVpp ID50 values versus UKNP1.18.1 HCVpp ID50 values is shown in FIG. 10B.

To assess possible associations between antigenicity and immunogenicity, correlations were calculated between measured binding affinity values for HMAbs and group immunogenicity measurements (endpoint titer or HCVpp ID50). Top correlations based on significance (p-value) are shown in FIG. 10C. As with the individual mouse correlation analysis noted above, HCVpp endpoint titers were excluded from this analysis due to insignificant binding values in several groups. Due to limited number of data points and limited overall variability in binding affinity measurements (FIG. 13), few correlations between antigenic and immunogenic parameters were highly significant, though binding of domain D HMAb HC84.26.WH.5DL was highly correlated with neutralization of J6 HCVpp (r=0.97, p=0.0003), as well as neutralization of UKNP1.18.1 HCVpp (r=0.88, p=0.008), while anticorrelations were detected for other antibody binding measurements (HEPC74 and HCV1) and HCVpp group neutralization values, at lower significance levels. The high correlations involving HMAb HC84.26.WH.5DL are not unexpected, based on the higher HMAb binding affinity to the H445P sE2 antigen and higher nAb responses induced by H445P; FIG. 10D compares UKNP1.18.1 neutralization with HC84.26.WH.5DL binding, where the point corresponding to H445P is in the upper right.

### 3. Discussion

In this study, a variety of rational design approaches were applied to engineer a modified HCV E2 glycoprotein to improve its antigenicity and immunogenicity. One of these approaches, removal of HVR1 (ΔHVR1), has been tested in several recent immunogenicity studies, in the context of E2 and E1E2. In this study, the E2 ΔHVR1 mutant with residues 384-407 removed, which retains residues 408-661 of E2 were tested; this is a more conservative truncation than previously tested ΔHVR1 mutants, in order to retain residue 408 which is binding determinant for the HC33.4 HMAb and others. Here this mutant was found to not be advantageous from an immunogenicity standpoint, which is in agreement with most other previous immunogenicity studies testing ΔHVR1 mutants. Although HVR1 is an immunogenic epitope, its removal from recombinant E2 glycoprotein does not appear to increase homologous or heterologous nAb titers, with the latter indicating that the level of antibodies targeting conserved nAb epitopes did not increase upon HVR1 removal. Removal of HVR1 is associated with increased nAb sensitivity and CD81 receptor binding, while HVR1 may modulate viral dynamics and open and closed conformations during envelope breathing. Despite its importance in the context of the virion and its dynamics, its removal appears to have a neutral or minimal effect on the immunogenicity of recombinant envelope glycoproteins.

Another design strategy tested in this study was hyperglycosylation, through structure-based addition of N-glycan sequons to mask antigenic domain A, which is associated with non-neutralizing antibodies. The concept of down-modulating immunity to this region was based on the observation that this region is highly immunogenic and may divert antibody responses to bNAb epitopes of lower immunogenicity. Through the efforts of isolating bNAbs to distinct regions on E2 from multiple HCV infected individuals, non-neutralizing antibodies to domain A are consistently identified. This strategy has been successfully employed for other glycoprotein immunogens, including for HIV Env SOSIP trimers, where the immunogenic V3 loop was masked with designed N-glycans. Surprisingly, some of the designs in this study exhibited an impact on recognition by antibodies targeting antigenic domain D on the front layer of E2, suggesting a possible interplay between the front and back layers of E2, as proposed previously based on global alanine scanning mutagenesis. As observed in the context of HIV Env, the designed E2 N-glycan variant tested for immunogenicity in this study (Y632NS) did not show improvements in nAb elicitation. However, its combination with ΔHVR1 did lead to modest improvement in nAb titers against one resistant isolate (UKNP2.4.1; p-value<0.05), compared with wild-type sE2. Previously we used insect cell expression to alter the N-glycan profile of sE2 versus mammalian cell expressed sE2 (Law J L M, et al., J Virol 92), and others have recently tested immunogenicity for glycan-deleted E2 and E1E2 variants; in neither case was a significant improvement in homologous and heterologous nAb responses observed for immunogens with altered glycans. Collectively, these results indicate that glycoengineering of E2 or E1E2 represents a more challenging, and possibly less beneficial, avenue for HCV immunogen design, however a report of success by others through insect cell expressed sE2 indicates that altered glycosylation may help in some instances.

The designed substitution H445P, which was generated to preferentially adopt the bnAb-bound form in a portion of E2 antigenic domain D that exhibits structural variability, showed the greatest level of success, both with regard to improvements in serum binding to homologous and heterologous HCVpp, as well as HCVpp neutralization of heterologous HCVpp. This design lies within a supersite of E2 associated with many broadly neutralizing antibodies, and through biophysical characterization and molecular dynamics simulation experiments, others have found that this region is likely quite flexible, providing a rationale for stabilizing key residues to engage and elicit bNAbs. Interestingly, a residue adjacent to the site of this design appears to be functionally important, with the Q444R substitution restoring viral infectivity in the context of an HCVpp with a domain E “glycan shift” substitution, N417S. The design strategy of utilizing proline residue substitutions to stabilize conformations of viral glycoproteins has been successful for HIV Env, respiratory syncytial virus (RSV) F, MERS coronavirus spike, and recently, the novel coronavirus (SARS-CoV-2) spike. The data from this study suggest that this approach is also useful in the context of HCV E2, and possibly E1E2.

This study provides a computational structure-based design of the HCV E2 glycoprotein to modulate its antigenicity and immunogenicity. Future studies with the H445P design can include testing of its antigenicity and immunogenicity in the context of HCV E1E2, testing immunogenicity in other animal models, as well as confirmation of its impact on E2 structure through high resolution X-ray structural characterization and additional biophysical characterization. Confirmation of improved elicitation of neutralizing antibodies with a cell-culture based HCV assay (HCVcc), versus the pseudoparticle-based assay (HCVpp) used in this study, can provide further insight into the impact of these and other HCV envelope glycoprotein variants. However, the employment of HCVpp does permit a greater ease in testing against clinical isolates. Furthermore, additional designed proline substitutions in this flexible E2 “neutralizing face” supersite may confer greater improvements in homologous and heterologous nAb elicitation; these can be generated using structure-based design, or with a semi-rational library-based approach, as was used to scan a large set of proline substitutions for HIV Env.

### 4. Materials and Methods

i. Computational Modeling and Design

Proline substitution designs to stabilize epitopes were modeled as previously described for design of T cell receptor binding loops, using a Ramachandran plot server to assess epitope residue backbone conformations for proline and pre-proline conformational similarities as well as explicit modeling of energetic effects of proline substitutions using the point mutagenesis mode of Rosetta version 2.3. N-glycan sequon substitutions (N×S, N×T) were modeled using Rosetta, followed by modeling of the N-glycan structure using the Glyprot web server. Assessment of residue side chain accessible surface areas was performed using NACCESS with default parameters.

ii. Protein and Antibody Expression and Purification

Expression and purification of recombinant soluble HCV E2 (sE2) and designs was performed. Briefly, the sequence from isolate H77C (GenBank accession number AF011751; residues 384-661) was cloned into the pSecTag2 vector (Invitrogen), transfected with 293fectin into FreeStyle HEK293-F cells (Invitrogen), and purified from culture supernatants by sequential HisTrap Ni²⁺-NTA and Superdex 200 columns (GE Healthcare). For recombinant HCV E1E2 expression, the H77C E1E2 glycoprotein coding region (GenBank accession number AF011751) was synthesized with a modified tPA signal peptide at the N-terminus and cloned into the vector pcDNA3.1+ at the cloning sites of KpnI/NotI (GenScript). Expi293 cells (Thermo Fisher) were used to express the E1E2 glycoprotein complex. In brief, the Expi293 cells were grown in Expi293 medium (ThermoFisher) at 37° C., 125 rpm, 8% CO2 and 80% humidity in Erlenmeyer sterile polycarbonate flasks (VWR). The day before the transfection, 2.0×10⁶ viable cells/ml was seeded in a flask and the manufacturer's protocol (A14524, ThermoFisher) was followed for transfection performance. After 72 hours post-transfection, the cell pellets were harvested by centrifuging cells at 3,000×g for 5 min and the cell pellet were then stored at −80° C. for further processing. Recombinant E1E2 was extracted from cell membranes using 1% NP-9 and purified via sequential Fractogel EMD TMAE (Millipore), Fractogel EMD SO₃⁻ (Millipore). HC84.26 immunoaffinity, and Galanthus Nivalis Lectin (GNL, Vector Laboratories) affinity chromatography. Monoclonal antibody HCV1 was provided by Dr. Yang Wang (MassBiologics, University of Massachusetts Medical School), and monoclonal antibodies AR3A, AR4A, and AR5A were provided by Dr. Mansun Law (Scripps Research Institute). All other monoclonal antibodies used in ELISA and binding studies were produced as previously described. A clone for mammalian expression of CD81 large extracellular loop (LEL), containing N-terminal tPA signal sequence and C-terminal twin Strep tag. CD81-LEL was expressed through transient transfection in Expi293F cells (ThermoFisher) and purified from supernatant with a Gravity Flow Strep-Tactin Superflow high capacity column (IBA Lifesciences). Purified CD81-LEL was polished by size exclusion chromatography (SEC) with a Superdex 75 10/300 GL column (GE Healthcare) on an Akta FPLC (GE Healthcare).

iii. ELISA Antigenic Characterization and Competition Assays

Cloning and characterization of E2 mutant antigenicity using ELISA was performed. Mutants were constructed in plasmids carrying the 1a H77C E1E2 coding sequence (GenBank accession number AF009606), as described previously. All the mutations were confirmed by DNA sequence analysis (Elim Biopharmaceuticals, Inc., Hayward, Calif.) for the desired mutations and for absence of unexpected residue changes in the full-length E1E2-encoding sequence. The resulting plasmids were transfected into HEK 293T cells for transient protein expression using the calcium-phosphate method. Individual E2 protein expression was normalized by binding of CBH-17, an HCV E2 HMAb to a linear epitope. Data are shown as mean values of two experiments performed in triplicate. Serum samples at specified dilutions were tested for their ability to block the binding of selected HCV HMAbs-conjugated with biotin in a GNA-captured E1E2 glycoproteins ELISA, as described (Keck Z Y et al., 2012, PLoS Pathog 8:e1002653). Data are shown as mean values of two experiments performed in triplicate.

iv. Biolayer Interferometry

The interaction of recombinant sE2 glycoproteins with CD81 and HMAbs in was measured using an Octet RED96 instrument and Ni²⁺-NTA biosensors (Pall ForteBio). The biosensors were loaded with 5 □g/mL of purified His6-tagged wild-type or mutant sE2 for 600 sec. Association for 300 sec followed by dissociation for 300 sec against a 2-fold concentration dilution series of each antibody was performed. Data analysis was performed using Octet Data Analysis 10.0 software and utilized reference subtraction at 0 nM antibody concentration, alignment to the baseline, interstep correction to the dissociation step, and Savitzky-Golay fitting. Curves were globally fitted based on association and dissociation to obtain K_{D} values.

v. Differential Scanning Calorimetry

Thermal melting curves for monomeric E2 proteins were acquired using a MicroCal PEAQ-DSC automated system (Malvern Panalytical). Purified monomeric E2 proteins were dialyzed into PBS prior to analysis and the dialysis buffer was used as the reference in the experiments. Samples were diluted to 10 □M in PBS prior to analysis. Thermal melting was probed at a scan rate of 90° C.·h⁻¹ over a temperature range of 25 to 115° C. All data analyses including estimation of the melting temperature were performed using standard protocols that are included with the PEAQ-DSC software.

vi. Mass Spectrometry

Digestion was performed on 40 μg each of HEK293-derived sE2 glycan sequon substitutions by denaturing using 6 M guanidine HCl, 1 mM EDTA in 0.1 M Tris, pH 7.8, reduced with a final concentration of 20 mM DTT (65° C. for 90 min), and alkylated at a final concentration of 50 mM iodoacetamide (room temperature for 30 min). Samples were then buffer exchanged into 1 M urea in 0.1 M Tris, pH 7.8 for digestion. Sequential digestion was performed using trypsin (1/50 enzyme/protein ratio, w/w) for 18 hours at 37° C., followed by chymotrypsin (1/20 enzyme:protein, w/w) overnight at room temperature. Samples were then absorbed onto Sep-Pak tC18 columns to remove proteolytic digestion buffer, eluted with 50% acetonitrile/0.1% trifluoroacetic acid (TFA) buffer and concentrated to dryness in a centrifugal vacuum concentrator. The samples were then resuspended in 50 mM Sodium acetate pH 4.5 and incubated with Endo F1, Endo F2, and Endo F3 (QA Bio) at 37° C. for 72 hours to remove complex glycans. LC-UV-MS analyses were performed using an UltiMate 3000 LC system coupled to an LTQ Orbitrap Discovery equipped with a heated electrospray ionization (HESI) source and operated in a top 5 dynamic exclusion mode. A volume of 25 μl (representing 10 μg of digested protein) of sample was loaded via the autosampler onto a C18 peptide column (AdvanceBio Peptide 2.7 um, 2.1×150 mm, Agilent part number 653750-902) enclosed in a thermostatted column oven set to 50° C. Samples were held at 4° C. while queued for injection. The chromatographic gradient was conducted as described previously. Identification of glycosylated peptides containing the glycan sequon substitution was performed using Byonic software and extracted ion chromatograms used for estimating the relative abundance of the glycosylated peptides in Byologic software (Protein Metrics).

vii. Animal Immunization

CD-1 mice were purchased from Charles River Laboratories. Prior to immunization, sE2 antigens were formulated with polyphosphazene adjuvant. Poly[di(carboxylatophenoxy)phosphazene], PCPP (molecular weight 800,000 Da) (Hadlock K G, et al., 2000, J Virol 74:1040716) was dissolved in PBS (pH 7.4) and mixed with sE2 antigen solution at 1:1 (prime) or 1:5 (w/w) (boost immunization) antigen:adjuvant ratio to provide for 50 mcg PCPP dose per animal. The absence of aggregation in adjuvanted formulations was confirmed by dynamic light scattering (DLS): single peak, z-average hydrodynamic diameter—60 nm. The formation of sE2 antigen—PCPP complex was proven by asymmetric flow field flow fractionation (AF4) as described (Andrianov A K, et al., 2004, Biomacromolecules 5:1999-2006). On scheduled vaccination days, groups of 6 female mice, age 7-9 weeks, were injected via the intraperitoneal (IP) route with a 50 μg sE2 prime (day 0) and boosted with 10 μg sE2 on days 7, 14, 28, and 42. Blood samples were collected prior to each injection with a terminal bleed on day 56. The collected samples were processed for serum by centrifugation and stored at −80° C. until analysis was performed.

viii. Serum Peptide and Protein ELISA

Domain-specific serum binding was tested using ELISA with C-terminal biotinylated peptides from H77C AS412 (aa 410-425; sequence NIQLINTNGSWHINST) and AS434 (aa 434-446; sequence NTGWLAGLFYQHK), using 2 μg/ml coating concentration. Recombinant sE2 and E1E2 proteins were captured onto GNA-coated microtiter plates. Endpoint titers were calculated by curve fitting in GraphPad Prism software, with endpoint OD defined as four times the highest absorbance value of Day 0 sera.

ix. HCV Pseudoparticle Generation

HCV pseudoparticles (HCVpp) were generated as described previously (19), by co-transfection of HEK293T cells with the murine leukemia virus (MLV) Gag-Pol packaging vector, luciferase reporter plasmid, and plasmid expressing HCV E1E2 using Lipofectamine 3000 (Thermo Fisher Scientific). Envelope-free control (empty plasmid) was used as negative control in all experiments. Supernatants containing HCVpp were harvested at 48 h and 72 h post-transfection, and filtered through 0.45 μm pore-sized membranes. Concentrated HCVpp were obtained by ultracentrifugation of 33 ml of filtered supernatants through a 7 ml 20% sucrose cushion using an SW 28 Beckman Coulter rotor at 25,000 rpm for 2.5 hours at 4° C.

x. HCVpp Serum Binding

For measurement of serum binding to HCVpp, 100 μL of 0.45 μm filtered HCVpp isolates were directly coated onto Nunc-immuno MaxiSorp (Thermo Scientific) microwells overnight at 4° C. Microwells were washed three times with 300 μL of 1×PBS, 0.05% Tween 20 in between steps. Wells were blocked with Pierce Protein-Free Blocking buffer (Thermo Scientific) for 1 hour. Serum sample dilutions made in the blocking buffer were added to the microwells and incubated for 1 hour at room temperature. Abs were detected with secondary HRP conjugated goat anti-mouse IgG H&L (Abcam, ab97023) and developed with TMB substrate solution (Bio-Rad). The reaction was stopped with 2N sulfuric acid. A Molecular Devices M3 plate reader was used to measure absorbance at 450 nm. Endpoint titers were calculated by curve fitting in GraphPad Prism software, with endpoint OD defined as four times the highest absorbance value of Day 0 sera.

xi. HCVpp Neutralization Assays

For infectivity and neutralization testing of HCVpp, 1.5×10⁴ Huh7 cells per well were plated in 96-well tissue culture plates (Corning) and incubated overnight at 37° C. The following day, HCVpp were mixed with appropriate amounts of antibody and then incubated for 1 h at 37° C. before adding them to Huh7 cells. After 72 h at 37° C., either 100 μl Bright-Glo (Promega) was added to each well and incubated for 2 min or cells were lysed with Cell lysis buffer (Promega E1500) and placed on a rocker for 15 min. Luciferase activity was then measured in relative light units (RLUs) using either a SpectraMax M3 microplate reader (Molecular Devices) with SoftMax Pro6 software (Bright-Glo protocol) or wells were individually injected with 50 μL luciferase substrate and read using a FLUOstar Omega plate reader (BMG Labtech) with MARS software. Infection by HCVpp was measured in the presence of anti-E2 MAbs, tested animal sera, pre-immune animal sera, and non-specific IgG at the same dilution. Each sample was tested in duplicate or triplicate. Neutralizing activities were reported as 50% inhibitory dilution (ID₅₀) values and were calculated by nonlinear curve fitting (GraphPad Prism), using lower and upper bounds (0% and 100% inhibition) as constraints to assist curve fitting.

xii. Statistical Comparisons

P-values between group endpoint titers and group ID₅₀ values were calculated using Kruskal-Wallis one-way analysis of variance (ANOVA), with Dunn's multiple comparison test, in Graphpad Prism software. Pearson correlations and correlation significance p-values were calculated in R (r-project.org).

Those skilled in the art will recognize, or be able to ascertain using no more than routine experimentation, many equivalents to the specific embodiments of the method and compositions described herein. Such equivalents are intended to be encompassed by the following claims.

We claim:

1. A modified hepatitis C virus (HCV) E2 glycoprotein comprising an antigenic domain D, wherein the modified hepatitis C virus (HCV) E2 glycoprotein comprises one or more amino acid alterations in the antigenic domain D.

2. The modified hepatitis C virus (HCV) E2 glycoprotein of claim 1, comprising an amino acid sequence with 70% identity to SEQ ID NO: 1.

3. The modified HCV E2 glycoprotein of claim 1 or 2, wherein at least one amino acid alteration is a proline substitution.

4. The modified HCV E2 glycoprotein of claim 3, wherein the proline substitution stabilizes an antibody-bound conformation of the antigenic domain D.

5. The modified HCV E2 glycoprotein of any of claims 3-4, wherein the proline substitution is a substitution of histidine with proline.

6. The modified HCV E2 glycoprotein of any one of claims 3-5, wherein the proline substitution occurs at a residue corresponding to position 445 of SEQ ID NO:1.

7. The modified HCV E2 glycoprotein of any one of claims 3-6, wherein the proline substitution corresponds to an H445P substitution as compared to SEQ ID NO:1.

8. The modified HCV E2 glycoprotein of any of claims 1-7, wherein the modified HCV E2 glycoprotein comprises the amino acid sequence of SEQ ID NO:2.

9. The modified HCV E2 glycoprotein of any of claims 1-6, wherein the modified HCV E2 glycoprotein comprises a sequence with 90% identity to SEQ ID NO:2, wherein the sequence comprises a H445P substitution as compared to SEQ ID NO:2.

10. The modified HCV E2 glycoprotein of any one of claims 1-9, wherein the antigenic domain D of the modified HCV E2 glycoprotein retains ability to bind to an antibody specific to the antigenic domain D.

11. The modified HCV E2 glycoprotein of claim 10, wherein the antibody is HC84.1 or HC84.26.

12. The modified HCV E2 glycoprotein of any one of claims 1-11, wherein the modified HCV E2 glycoprotein is soluble.

13. The modified HCV E2 glycoprotein of any one of claims 1-12, wherein the HCV E2 glycoprotein further comprises an antigenic domain A, wherein the antigenic domain A comprises an N-glycan sequon substitution.

14. A modified hepatitis C virus (HCV) E2 glycoprotein comprising an antigenic domain A, wherein the antigenic domain A comprises an N-glycan sequon substitution.

15. The modified HCV E2 glycoprotein of claim 14, wherein the N-glycan sequon substitution results in an Asn-Xaa-Ser or Asn-Xaa-Thr substitution, wherein Xaa is any amino acid except proline.

16. The modified hepatitis C virus (HCV) E2 glycoprotein of any of claims 14-15, comprising an amino acid sequence with 70% identity to SEQ ID NO: 1.

17. The modified HCV E2 glycoprotein of any one of claims 14-16, wherein the N-glycan sequon substitution occurs at a residue corresponding to positions 632 and 634 of SEQ ID NO:1.

18. The modified HCV E2 glycoprotein of claim 17, wherein the N-glycan sequon substitution is Y632N-G634S as compared to SEQ ID NO:1.

19. The modified HCV E2 glycoprotein of claim 18 comprising the amino acid sequence of SEQ ID NO:3.

20. The modified HCV E2 glycoprotein of any one of claims 14-16, wherein the N-glycan sequon substitution occurs at a residue corresponding to positions 630 and 632 of SEQ ID NO:1.

21. The modified HCV E2 glycoprotein of claim 20, wherein the N-glycan sequon substitution is R630N-Y632T as compared to SEQ ID NO:1.

22. A composition comprising one or more of the modified HCV E2 glycoproteins of claims 1-21 and a pharmaceutically acceptable carrier thereof.

23. A method of increasing HCV E2 glycoprotein antigenicity in a subject in need thereof comprising administering a composition comprising one or more of the modified HCV E2 glycoproteins of any of claims 1-13, wherein the increase in HCV E2 glycoprotein antigenicity is an increase in antigenic domain D antigenicity.

24. A method of decreasing HCV E2 glycoprotein antigenicity in a subject in need thereof comprising administering a composition comprising one or more of the modified HCV E2 glycoproteins of claims 14-20, wherein the decrease in HCV E2 glycoprotein antigenicity is a decrease in antigenic domain A antigenicity.

25. A method of inducing an immune response in a subject in need thereof comprising administering to the subject in need thereof a composition comprising one or more of the modified HCV E2 glycoproteins of claims 1-13.

26. The method of any of claims 23-25, wherein the subject in need thereof has been infected with hepatitis C virus (HCV) or is at risk for being infected with HCV.

27. A method of treating a subject having HCV comprising administering to the subject a composition comprising one or more of the modified HCV E2 glycoproteins of claims 1-21.

28. The method of claim 27, wherein the modified HCV E2 glycoprotein induces an immune response against HCV in the subjects.

29. A method of generating neutralizing antibodies (nAbs) to the antigenic domain D of HCV in a subject in need thereof comprising administering to the subject in need thereof a composition comprising one or more of the modified HCV E2 glycoproteins of claims 1-20.

30. A method for immunizing a subject comprising: administering to the subject a composition comprising one or more of the modified HCV E2 glycoproteins of claims 1-20.

31. The method of claim 30, wherein a protective immune response effective to reduce or eliminate subsequent HCV-infection clinical signs in the subject, relative to a non-immunized control subject of the same species, is elicited by administration of the composition.

32. The method of claim 30, wherein a protective immune response effective to reduce HCV infection risk in the subject, relative to a non-immunized control subject of the same species, is elicited by administration of the composition.

33. The method of claim 30, wherein the composition is administered subcutaneously, intramuscularly, orally, or via spray.


SEQUENCE LISTING (9 sequences)

SEQ ID NO: 1 - protein, 363 aa, Hepatitis C virus
ETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 2 - protein, 363 aa, Artificial Sequence
  synthetic construct; modified E2
ETHVTGGSAGRTTAGLVGLLTPGAKQNIQLINTNGSWHINSTALNCNESLNTGWLAGLFYQPKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 3 - protein, 278 aa, Artificial Sequence
  synthetic construct; soluble E2
ETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSE

SEQ ID NO: 4 - protein, 278 aa, Artificial Sequence
  synthetic construct; modified soluble E2
ETHVTGGSAGRTTAGLVGLLTPGAKQNIQLINTNGSWHINSTALNCNESLNTGWLAGLFYQPKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSE

SEQ ID NO: 5 - protein, 363 aa, Artificial Sequence
  synthetic construct; modified E2
ETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMNVSGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 6 - protein, 470 aa, Artificial Sequence
  synthetic construct; E1E2 heterodimer
YQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSE

SEQ ID NO: 7 - protein, 470 aa, Artificial Sequence
  synthetic construct; modified E1E2 heterodimer
YQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGSAGRTTAGLVGLLTPGAKQNIQLINTNGSWHINSTALNCNESLNTGWLAGLFYQPKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSE

SEQ ID NO: 8 - protein, 470 aa, Artificial Sequence
  synthetic construct; modified E1E2 heterodimer
YQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMNVSGVEHRLEAACNWTRGERCDLEDRDRSE

SEQ ID NO: 9 - protein, 278 aa, Artificial Sequence
  synthetic construct; modified E2
ETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMNVSGVEHRLEAACNWTRGERCDLEDRDRSE